(19) United States
(12) Patent Application Publication
(10) Pub. No.: US 2012/0183605 A1
(43) Pub. Date: Jul. 19, 2012

(54) QUININE FORMULATIONS, METHOD OF MAKING, AND METHOD OF USE THEREOF

(76) Inventors: Kristin Arnold, Morrisville, PA (US); Matthew W. Davis, Erwinna, PA (US); Suman Wason, Basking Ridge, NJ (US); Siva Rama K. Nutalapati, Princeton, NJ (US)

(21) Appl. No.: 13/232,581

(22) Filed: Sep. 14, 2011

Related U.S. Application Data
(63) Continuation-in-part of application No. 12/971,051, filed on Dec. 17, 2010.
Application No. 12/971,051 is a continuation-in-part of application No. 12/731,315, filed on Mar. 25, 2010.
(60) Provisional application No. 61/287,747, filed on Dec. 18, 2009.

(51) Int. Cl. A61K 9/48; A61K 31/439; A61P 33/00; A61K 9/00

(52) U.S. Cl. 424/451; 424/400; 514/305

(57) ABSTRACT

Disclosed herein are quinine formulations and methods of using quinine formulations. Specifically disclosed herein are solid oral dosage forms which can be administered as a capsule or tablet, or alternatively as a sprinkle form with the patient experiencing little or no bitter taste. The dosage forms provide immediate release in vitro and in vivo.

### CROSS REFERENCE TO RELATED APPLICATION

This application is a continuation-in-part of U.S. application Ser. No. 12/971,051 filed Dec. 17, 2010, and a continuation-in-part of U.S. application Ser. No. 12/731,315 filed Mar. 25, 2010, each of which claims the benefit of U.S. Provisional Application Ser. No. 61/287,747 filed Dec. 18, 2009; and each of which is hereby incorporated by reference in its entirety.

### BACKGROUND

Malaria is a parasitic disease caused by the Plasmodium species P. falciparum, P. vivax, P. ovale and P. malariae. The malaria parasite causes intermittent fevers and chills. It affects multiple organs and systems, including red blood cells, the kidneys, liver, spleen and brain. It is estimated by the World Health Organization (WHO) that up to 500 million persons per year are infected with malaria, with 200 to 300 million people suffering from malaria at any given time (See Roll Back Malaria. World Health Organization. available at: www.rbm.who.int/cmc_upload/0/000/015372/RBMInfosheet_{—}1.htm). Up to 3 million will die each year. If P. falciparum infection goes untreated or is not treated appropriately, general observations indicate that mortality is high, killing up to 25% of non-immune adults within 2 weeks of a primary attack [Taylor T E, Strickland G T. Malaria. In: Strickland G T, ed. Hunter's Tropical Medicine and Emerging Infectious Diseases. 8th ed. Philadelphia, Pa.: W.B. Saunders Company; 2000.] A significant number of these cases are found in Central America, South America, Asia, and Africa. Known antimalarial agents include 9-aminoacridines (e.g. mepacrine), 4-aminoquinolines (e.g. amodiaquine, chloroquine, hydroxychloroquine), 8-aminoquinolines (e.g. primaquine, quinocide), biguanides with an inhibiting effect on dihydrofolic acid reductase (e.g. chlorproguanil, cycloguanil, proguanil), diaminopyrimidines (e.g. pyrimethamine), quinine salts, sulphones such as dapsone, sulphonamides, sulphanilamides and antibiotics such as tetracycline.

Quinine (cinchonan-9-ol, 6′-methoxy-, (8α,9R)-) is an antiprotozoal and an antimyotonic, and is known for the treatment of malaria caused by Plasmodium species, the treatment and prophylaxis of nocturnal recumbency leg muscle cramps, and the treatment of babesiosis caused by Babesia microti.

Quinine is extremely bitter, thus making patient compliance difficult if even small amounts of quinine are present on the surface of oral dosage forms. A currently available form of quinine sulfate is powdered quinine sulfate in a capsule which provides a sufficient barrier so that the patient does not taste the quinine when the capsule is administered. However, certain populations of patients, such as the elderly and pediatric patients, have difficultly swallowing solid, oral dosage forms such as tablets and capsules due to their large size.

There remains a need in the art for a single, versatile oral quinine formulation that can be administered to patients that have the ability to swallow traditionally sized dosage forms which at the same time can be administered to patient populations that have difficulty swallowing traditionally sized dosage forms. Such a formulation should exhibit an acceptable taste profile in order to improve patient compliance and acceptability.

### SUMMARY

In one embodiment, a quinine sprinkle formulation comprises a plurality of coated subunits, wherein each coated subunit comprises a core subunit comprising quinine or a pharmaceutically acceptable salt thereof, and a pharmaceutically acceptable excipient, and a coating on the outside of the core subunit, wherein the coating comprises a polymeric coating material, wherein the polymeric coating material is chitosan; ethylcellulose; hydroxypropyl methylcellulose acetate succinate; cellulose acetate phthalate; a (meth)acrylic acid copolymer; hydroxypropyl methylcellulose succinate; cellulose acetate succinate; cellulose acetate hexahydrophthalate; hydroxypropyl methylcellulose hexahydrophthalate; hydroxypropyl methylcellulose phthalate; cellulose propionate phthalate; cellulose acetate maleate; cellulose acetate trimellitate; cellulose acetate butyrate; cellulose acetate propionate; a polyvinylacetate phthalate; zein; or a combination thereof; optionally in combination with a plasticizer, a stabilizer, a water-soluble component, an anti-tacking agent, a surfactant, or a combination thereof; wherein the quinine formulation exhibits immediate-release profile; and wherein the quinine sprinkle formulation forms a palatable mixture with applesauce for up to one hour after adding the plurality of coated subunits in applesauce.

In another embodiment, a method of administering quinine comprises administering a quinine sprinkle formulation to a patient for treatment of uncomplicated Plasmodium falciparum malaria, treatment of severe or complicated Plasmodium falciparum malaria, treatment of Plasmodium vivax infection, treatment of babesiosis caused by Babesia microti, or prevention of malaria; wherein the quinine sprinkle formulation comprises a plurality of coated subunits, wherein each coated subunit comprises a core subunit comprising quinine or a pharmaceutically acceptable salt thereof, and a pharmaceutically acceptable excipient, and a coating on the outside of the core subunit, wherein the coating comprises a polymeric coating material, wherein the polymeric coating material is chitosan; ethylcellulose; hydroxypropyl methylcellulose acetate succinate; cellulose acetate phthalate; a (meth)acrylic acid copolymer; hydroxypropyl methylcellulose succinate; cellulose acetate succinate; cellulose acetate hexahydrophthalate; hydroxypropyl methylcellulose hexahydrophthalate; hydroxypropyl methylcellulose phthalate; cellulose propionate phthalate; cellulose acetate maleate; cellulose acetate trimellitate; cellulose acetate butyrate; cellulose acetate propionate; a polyvinylacetate phthalate; zein; or a combination thereof; optionally in combination with a plasticizer, a stabilizer, a water-soluble component, an anti-tacking agent, a surfactant, or a combination thereof; wherein the quinine formulation exhibits immediate-release profile; and wherein the quinine sprinkle formulation is a palatable mixture for up to one hour after preparation of the mixture, the palatable mixture comprising the plurality of coated subunits sprinkled in applesauce.

In yet another embodiment, a quinine formulation comprises a solid oral dosage form comprising a plurality of coated subunits, wherein each coated subunit comprises a core subunit comprising quinine or a pharmaceutically acceptable salt thereof, and a pharmaceutically acceptable excipient, and a coating on the outside of the core subunit, wherein the coating comprises a polymeric coating material, wherein the polymeric coating material is chitosan; ethylcellulose; hydroxypropyl methylcellulose acetate succinate; cellulose acetate phthalate; a (meth)acrylic acid copolymer; hydroxypropyl methylcellulose succinate; cellulose acetate succinate; cellulose acetate hexahydrophthalate; hydroxypropyl methylcellulose hexahydrophthalate; hydroxypropyl methylcellulose phthalate; cellulose propionate phthalate; cellulose acetate maleate; cellulose acetate trimellitate; cellulose acetate butyrate; cellulose acetate propionate; a polyvinylacetate phthalate; zein; or a combination thereof; optionally in combination with a plasticizer, a stabilizer, a water-soluble component, an anti-tacking agent, a surfactant, or a combination thereof; wherein the quinine formulation exhibits immediate-release profile; and wherein the quinine formulation can be administered as a single unit solid oral dosage form or administered as a sprinkle on food.

Also disclosed herein are methods of treating and methods of reducing or eliminating incidents of gastric upset and irritation experienced by the administration of capsule formulations of powdered quinine without food.

### DETAILED DESCRIPTION

Disclosed herein are immediate-release solid, oral quinine formulations which offer the flexibility of either being orally administered as a single unit (e.g., capsule or tablet form ingested whole) or as a sprinkle form onto food (either prepared as a sachet or by opening a capsule or crushing a tablet). The immediate-release solid, oral quinine formulation upon administration results in minimal or no bitter taste experienced by the patient. Additionally, when administered as a sprinkle, the patient experiences minimal or no bitter taste for up to an hour or more from the time of preparing the sprinkle formulation with food, specifically up to an hour from the time of preparing the sprinkle formulation with food.

The quinine formulation is generally an immediate-release multiparticulate system containing subunits comprising quinine. “Subunit” includes a minitablet, a bead, a spheroid, a microsphere, a seed, a pellet, a caplet, a microcapsule, a granule, and the like that can provide an oral dosage form alone or when combined with other subunits. The formulation comprises a capsule comprising a plurality of coated subunits, wherein each coated subunit comprises a core subunit comprising quinine or a pharmaceutically acceptable salt thereof and a pharmaceutically acceptable excipient; and a coating on the outside of the core subunit. The quinine sulfate is taste-masked by the coating. When used as a sprinkle, the formulation provides acceptable taste-masking for a sufficient period of time in a chosen food or liquid vehicle of administration, whether acidic, neutral, or basic.

The quinine formulations provide immediate release of quinine in vivo and when tested in an in vitro dissolution test.

The plurality of coated subunits of the multiparticulate system can be loaded into hard or soft capsule shells, specifically gelatin capsules, compressed into crushable tablets, or prepared as a sachet. When administered as a sprinkle formulation over food such as applesauce, the capsules can merely be twisted or broken open and the coated subunits added to the food without breaking the multiparticles. Alternatively, the tablets containing the multiparticulate system can be crushed with a low force (e.g., finger crushable) to release the multiparticulate system without damaging the coating. The quinine sulfate is taste-masked by the coating on the subunits. The taste-masking is effective in a variety of foods of varying pH, but particularly in low acid food such as applesauce. The pH of applesauce is about 4.0 or lower, typically about 3.4 to about 4.0. Thus the coating over the subunits function as a taste-masking coating even at a low pH while at the same time not delaying or extending the release of the active agent from the formulation. It was surprisingly found that certain coating polymers known as sustained-release, delayed-release, extended-release, or pulse-release coatings can be used to provide suitable taste-masking of quinine yet at the same time result in an immediate-release quinine dosage formulation.

The coated subunit comprises a core subunit comprising quinine and a pharmaceutically acceptable excipient, and a coating on the outside of the core subunit, wherein the coating comprises a polymeric coating material.

“Quinine” as used herein is inclusive of all pharmaceutically acceptable salt forms, crystalline forms, amorphous form, polymorphic forms, solvates, and hydrates unless specifically indicated otherwise. As used herein, “quinine sulfate” means cinchonan-9-ol, 6′-methoxy-, (8a,9R)-, sulfate (2:1) or cinchonan-9-ol, 6′-methoxy-, (8a,9R)-, sulfate (2:1) dihydrate unless otherwise indicated.

“Pharmaceutically acceptable salts” include derivatives of the active agent (e.g. quinine), wherein the parent compound is modified by making acid addition salts thereof, and further refers to pharmaceutically acceptable solvates, including hydrates, of such compounds and such salts. Also included are all crystalline, amorphous, polymorph, and co-crystal forms. Examples of pharmaceutically acceptable salts include, but are not limited to, mineral or organic acid addition salts; and the like, and combinations comprising one or more of the foregoing salts. The pharmaceutically acceptable salts include non-toxic salts, for example, from non-toxic inorganic or organic acids. For example, non-toxic acid salts include those derived from inorganic acids such as hydrochloric, hydrobromic, sulfuric, sulfamic, phosphoric, nitric and the like. Pharmaceutically acceptable organic salts includes salts prepared from organic acids such as acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Specific quinine salts include quinine sulfate, quinine hydrochloride, quinine dihydrochloride, and hydrates, solvates, or polymorphic forms thereof.

As used herein, “pharmaceutically acceptable excipient” means any other component added to the pharmaceutical formulation other than the active agent. Excipients may be added to facilitate manufacture, enhance stability, enhance product characteristics, enhance bioavailability, enhance patient acceptability, etc. Pharmaceutical excipients include carriers, fillers, binders, disintegrants, lubricants, glidants, granulating agent, compression aids, colors, sweeteners, preservatives, suspending agents, dispersing agents, film formers, flavors, printing inks, buffer agents, pH adjusters, preservatives etc. In some instances, a single material will meet two or more of the foregoing general classifications.

Exemplary pharmaceutically acceptable excipients include fillers, such as a water insoluble filler, water soluble filler, or a combination comprising at least one of the foregoing. The filler may be a water insoluble filler, such as carnauba wax, stearic acid, silicon dioxide, titanium dioxide, talc, alumina, starch, kaolin, polacrilin potassium, powdered cellulose, microcrystalline cellulose, sodium citrate, dicalcium phosphate or a combination comprising at least one of the foregoing fillers. Exemplary water-soluble fillers include water soluble sugars and sugar alcohols, specifically lactose, glucose, fructose, sucrose, mannose, dextrose, galactose, the corresponding sugar alcohols and other sugar alcohols, such as mannitol, sorbitol, xylitol, or a combination comprising at least one of the foregoing fillers.

Exemplary binders include alginic acid, a carbomer, carboxymethylcellulose calcium, carboxymethylcellulose sodium, carrageenan, cellulose acetate phthalate, chitosan, ethyl cellulose, guar gum, hydroxyethyl cellulose, hydroxyethylmethyl cellulose, hydroxypropyl cellulose, hydroxypropyl methyl cellulose, methyl cellulose, microcrystalline cellulose, poloxamer, polyethylene oxide, polymethacrylates, povidone, a saccharide, starch, partially pregelatinized starch, and the like, or a combination comprising at least one of the foregoing binders.

Exemplary disintegrants include alginic acid, carboxymethylcellulose calcium, carboxymethylcellulose sodium, cross-linked sodium carboxymethylcellulose (sodium croscarmellose), powdered cellulose, chitosan, croscarmellose sodium, crospovidone, guar gum, low substituted hydroxypropyl cellulose, methyl cellulose, microcrystalline cellulose, sodium alginate, sodium starch glycolate, partially pregelatinized starch, pregelatinized starch, starch, sodium carboxymethyl starch, and the like, or a combination comprising at least one of the foregoing disintegrants.

Exemplary lubricants include calcium stearate, magnesium stearate, glyceryl behenate, glyceryl palmitostearate, hydrogenated castor oil, light mineral oil, sodium lauryl sulfate, magnesium lauryl sulfate, sodium stearyl fumarate, stearic acid, zinc stearate, or a combination comprising at least one of the foregoing lubricants.

Exemplary glidants include colloidal silica, amorphous silica, precipitated silica, talc, calcium phosphate tribasic, calcium silicate, magnesium silicate, magnesium trisilicate, or a combination comprising at least one of the foregoing, and the like.

It is noted that the pharmaceutically acceptable excipients are used in an amount that does not delay or prolong the release of the quinine from the formulation such that the formulation can no longer be defined as immediate-release.

The core subunits may be prepared by, for example, dry granulation or wet granulation followed by compression or compaction, melt extrusion and spheronization, layering (e.g., spray layering suspension or solution), and the like. Examples of such techniques include direct compression, using appropriate punches and dies, the punches and dies are fitted to a suitable rotary tableting press; injection or compression molding using suitable molds fitted to a compression unit, granulation followed by compression; and extrusion in the form of a paste, into a mold or to an extrudate to be cut into lengths.

The core subunits can be prepared by compression into a compressed form (e.g., minitablets) using conventional tableting equipment using standard techniques. Techniques and compositions for making tablets (compressed and molded) are described in Remington's Pharmaceutical Sciences, (Aurther Osol., editor), 1553-1593 (1980).

Layering techniques suitable to prepare the core subunits include coating inert cores with a layering solution or dispersion of quinine and a pharmaceutically acceptable excipient. Repeated layering can be used to build the subunit size and increase active agent amount.

Exemplary liquids that can be used to prepare the layering dispersion or solution for the layering technique include water, lower alkyl alcohols (e.g., methanol, ethanol, n-propanol, isopropanol, etc.), lower alkyl ketones or acetates (e.g., acetone, ethyl acetate, etc.), lower alkyl ethers (e.g., ethyl ether, tetrahydrofuran, etc.), acetonitrile, lower halogenated alkyls (e.g., dichloromethane, etc.), or a combination comprising at least one of the foregoing solvents.

Materials suitable for use as the inert cores upon which layers containing quinine and a pharmaceutically acceptable excipient are applied onto include pharmaceutically acceptable materials that have appropriate dimensions and firmness. Examples of such materials are polymers e.g. plastic resins; inorganic substances, e.g. silica, glass, hydroxyapatite, salts (sodium or potassium chloride, calcium or magnesium carbonate) and the like; organic substances, e.g. activated carbon, acids (citric, fumaric, tartaric, ascorbic and the like acids), and saccharides and derivatives thereof. The saccharides include sugars, oligosaccharides, polysaccharides and their derivatives, for example, glucose, rhamnose, galactose, lactose, sucrose, mannitol, sorbitol, dextrin, maltodextrin, cellulose, microcrystalline cellulose, sodium carboxymethyl cellulose, starches (maize, rice, potato, wheat, tapioca) and the like.

The inert core can have an average diameter of about 250 to about 2500 micrometers, specifically about 500 to about 2000 micrometers, and yet more specifically about 750 to about 1500 micrometers.

To achieve sufficient taste masking of the quinine, the diameter of the core subunits are sufficiently large to allow for an even coat of the polymeric coating material to prevent leaks and to control taste leak through.

In one embodiment, the core subunits specifically have an average diameter of about 500 to about 4000 micrometers, specifically about 1000 to about 3500 micrometers, yet more specifically about 1500 to about 3250 micrometers, more specifically about 1750 to about 3000 micrometers, and still yet more specifically about 2000 to about 2500 micrometers.

In one embodiment, the core subunits are minitablets having an average length of its longest dimension of about 500 to about 4000 micrometers, specifically about 1000 to about 3500 micrometers, yet more specifically about 1500 to about 3250 micrometers, more specifically about 1750 to about 3000 micrometers, and still yet more specifically about 2000 to about 2500 micrometers.

Each subunit can contain any amount of quinine or salt thereof up to about 99 wt %, specifically about 10 to about 98 wt %, more specifically about 25 to about 95 wt %, yet more specifically about 50 to about 90 wt %, and still yet more specifically about 75 to about 85 wt % based on the total weight of the uncoated subunit.

In one embodiment, the amount of quinine sulfate per subunit is about 5 to about 12 mg, specifically about 7 to about 10 mg.

In one embodiment, the polymeric coating material used to coat the core subunits provide adequate taste-masking without delaying or extending the release of the active agent from the formulation. The polymeric coating material can be selected from a polymer that, when coated on a subunit, does not significantly dissolve in the substance used in the sprinkle form, or the saliva of the patient, yet will dissolve in the gastric juice of the stomach of the patient to provide immediate-release of the active agent. Surprisingly, in some instances, the polymeric coating materials used to coat the core subunits are controlled- or extended release polymers that provide adequate taste masking without delaying or extending the release of the active agent.

Suitable polymeric coating material for use to prepare the coated core subunits include chitosan, ethylcellulose, (e.g. ethylcellulose, such as AQUACOAT, a 30% dispersion available from FMC, Philadelphia, Pa.; SURELEASE a 25% dispersion further containing a stabilizer or other coating component (e.g., ammonium oleate, dibutyl sebacate, colloidal anhydrous silica, medium chain triglycerides, etc.) available from Colorcon, West Point, Pa.; Ethocel; or Aqualon) optionally combined with a water-soluble component (e.g., a hydroxyalkyl(alkylcellulose); hydroxypropyl methylcellulose acetate succinate (HPMCAS); cellulose acetate phthalate (CAP) optionally combined with a water-soluble component; a (meth)acrylic acid copolymer; hydroxypropyl methylcellulose succinate; cellulose acetate succinate; cellulose acetate hexahydrophthalate; hydroxypropyl methylcellulose hexahydrophthalate; hydroxypropyl methylcellulose phthalate (HPMCP); cellulose propionate phthalate; cellulose acetate maleate; cellulose acetate trimellitate; cellulose acetate butyrate; cellulose acetate propionate; a poly(meth)acrylic acid; a poly(meth)acrylate; a polyvinylacetate phthalate; zein; and the like, or a combination comprising at least one of the foregoing materials. “(Meth)acrylic or (meth)acrylate” is inclusive of acrylic, methacrylic, acrylate, or methacrylate.

Exemplary polymethacrylates include copolymers of acrylic and methacrylic acid esters, such as a. an aminomethacrylate copolymer USP/NF such as a poly(butyl methacrylate, (2-dimethyl aminoethyl)methacrylate, methyl methacrylate) 1:2:1 (e.g., EUDRAGIT E 100, EUDRAGIT EPO, and EUDRAGIT E 12.5; CAS No. 24938-16-7); b. a poly(methacrylic acid, ethyl acrylate) 1:1 (e.g., EUDRAGIT L30 D-55, EUDRAGIT L100-55, EASTACRYL 30D, KOLLICOAT MAE 30D AND 30DP; CAS No. 25212-88-8); c. a poly(methacrylic acid, methyl methacrylate) 1:1 (e.g., EUDRAGIT L 100, EUDRAGIT L 12.5 and 12.5 P; also known as methacrylic acid copolymer, type A NF; CAS No. 25806-15-1); d. a poly(methacrylic acid, methyl methacrylate) 1:2 (e.g. EUDRAGIT S 100, EUDRAGIT S 12.5 and 12.5P; CAS No. 25086-15-1); e. a poly(ethyl acrylate, methylmethacrylate, trimethylammonioethyl methacrylate chloride) 1:2:0.2 or 1:2:0.1 (e.g., EUDRAGITS RL 100, RL PO, RL 30 D, RL 12.5, RS 100, RS PO, RS 30 D, or RS 12.5; CAS No. 33434-24-1); f. a poly(ethyl acrylate, methyl methacrylate) 2:1 (e.g. EUDRAGIT NE 30 D; CAS No. 9010-88-2); and the like, or a combination comprising at least one of the foregoing materials.

Specific polymeric coating material include a combination of ethylcellulose and hydroxypropyl methylcellulose; a combination of cellulose acetate phthalate and hydroxypropyl methylcellulose; a poly(butyl methacrylate, (2-dimethyl aminoethyl)methacrylate, methyl methacrylate) 1:2:1; or a poly(methacrylic acid, ethyl acrylate) 1:1.

In addition to the polymeric coating material, the coating can optionally contain additional pharmaceutically acceptable excipients such as a plasticizer, a stabilizer, a water-soluble component (e.g. pore formers), an anti-tacking agent (e.g., talc), a surfactant, and the like, a combination comprising at least one of the foregoing. The water-soluble component can be an agent that can form channels through the coating upon the hydration or dissolution of the water-soluble component. Specifically, the water-soluble component can be a hydroxyalkylcellulose, hydroxyalkyl(alkylcellulose), carboxymethylcellulose, salts thereof, or a combination comprising at least one of the foregoing. Particular examples of these water-soluble components include hydroxyethylcellulose, hydroxypropylcellulose, hydroxyethyl methylcellulose, hydroxypropyl methylcellulose, carboxymethylcellulose, sodium carboxymethylcellulose, or a combination comprising at least one of the foregoing materials. Other exemplary water-soluble materials include a povidone; a saccharide (e.g., lactose, and the like); a metal stearate; an inorganic salt (e.g., dibasic calcium phosphate, sodium chloride, and the like); a polyethylene glycol (e.g., polyethylene glycol (PEG) 1450, and the like); a sugar alcohol (e.g., sorbitol, mannitol, and the like); an alkali alkyl sulfate (e.g., sodium lauryl sulfate); a polyoxyethylene sorbitan fatty acid ester (e.g., polysorbate); methyacrylate copolymers (e.g., EUDRAGIT® RL); or a combination comprising at least one of the foregoing pore forming materials.

The weight ratio of polymeric coating material to water-soluble component in the coating can be about 10:1 to about 1:10, specifically about 5:1 to about 1:5, more specifically about 3:1 to about 1:3, yet more specifically about 2:1 to about 1:2, and still more specifically about 1:1. In one embodiment, the polymeric coating material consists essentially of ethyl cellulose and the water-soluble component is hydroxypropyl methyl cellulose. In another embodiment, the polymeric coating material consists essentially of cellulose acetate phthalate and the water-soluble component is hydroxypropyl methyl cellulose. In the two foregoing embodiments, other components such as a plasticizer, a stabilizer, an anti-tacking agent, a surfactant, or a combination thereof can be present, but no other polymeric coating material is present.

The inclusion of an effective amount of a plasticizer in the coating can improve the physical properties of the coating. For example, because ethylcellulose has a relatively high glass transition temperature and does not form flexible films under normal coating conditions, it may be advantageous to add plasticizer to the ethylcellulose before using the same as a coating material. Generally, the amount of plasticizer included in a coating solution is based on the concentration of the polymer, e.g., most often from about 1 wt % to about 50 wt % of the polymer. Concentrations of the plasticizer, however, can be determined by routine experimentation.

Examples of plasticizers for ethyl cellulose and other celluloses include dibutyl sebacate, diethyl phthalate, triethyl citrate, tributyl citrate, triacetin, or a combination comprising at least one of the foregoing plasticizers; although it is possible that other water-insoluble plasticizers (such as acetylated monoglycerides, phthalate esters, castor oil, etc.) can be used.

Examples of plasticizers for (meth)acrylic/(meth)acrylate polymers include citric acid esters such as triethyl citrate NF, tributyl citrate, dibutyl phthalate, 1,2-propylene glycol, polyethylene glycols, propylene glycol, diethyl phthalate, castor oil, triacetin, stearic acid, or a combination comprising at least one of the foregoing plasticizers; although it is possible that other plasticizers (such as acetylated monoglycerides, phthalate esters, castor oil, etc.) can be used.

Suitable methods can be used to apply the coating material to the surface of the subunits. Processes such as simple or complex coacervation, interfacial polymerization, liquid drying, thermal and ionic gelation, spray drying, spray chilling, fluidized bed coating, pan coating, or electrostatic deposition may be used.

To obtain taste-masking of the quinine in a manner sufficient to allow sprinkle formulations in foods of a variety of pH, each subunit can be coated with an amount of polymeric coating material and optional water-soluble component, in an amount of about 1 wt % to about 30 wt %, specifically about 3 wt % to about 20 wt %, more specifically about 4 wt % to about 12 wt %, and yet more specifically about 6 wt % to about 10 wt % based on the total weight of the core subunit, polymeric coating material and optional water-soluble component; although the amounts can be greater or lesser depending upon the composition of the core subunit, size of the core subunit, amount of plasticizer or surfactant, among other things.

When coating, the weight gain of the polymeric coating material and optional water-soluble component can be in an amount of about 1 to about 30% weight gain based on the weight of the core subunit, specifically about 3 to about 20%, more specifically about 4 to about 12%, and yet more specifically about 6 to about 10% weight gain based on the total weight of the core subunit, polymeric coating material and optional water-soluble component; although the amounts can be greater or lesser depending upon the composition of the core subunit, size of the core subunit, amount of plasticizer or surfactant, among other things.

In certain embodiments, an optional intermediate coating is used between the core subunit and the coating providing taste-masking properties. Such an intermediate coating can be used to protect the active agent or other component of the core subunit from the material used in the taste-masking coating. Exemplary intermediate coatings include film forming polymers such as hydroxyethyl cellulose, hydroxypropyl cellulose, gelatin, hydroxypropyl methylcellulose, polyethylene glycol, polyethylene oxide, and the like, or a combination comprising at least one of the foregoing; and a plasticizer.

In one embodiment, the plurality of coated subunits of the multiparticulate system can be loaded into hard or soft capsule shells (e.g., gelatin capsules) using techniques well-known in the art.

In another embodiment, the plurality of coated subunits of the multiparticulate system is prepared as a sachet using techniques well-known in the art.

In still yet another embodiment, the plurality of coated subunits of the multiparticulate system can be mixed with an appropriate excipient and compressed into crushable tablets. The tablet can either be administered whole or lightly crushed, such as with finger pressure, to release the individual coated subunits and sprinkled over an appropriate vehicle (e.g., applesauce). The crushable tablet can be prepared using direct compression processes and excipients with care taken in the process to avoid damaging the coating of the individual subunits. Suitable excipients to prepare the crushable tablet include those typically used for chewable tablets including mono- and di-saccharides, sugar polyols, and the like, or a combination comprising at least one of the foregoing. Exemplary excipients include mannitol, sorbitol, xylitol, maltitol, lactose, sucrose, maltose or a combination comprising at least one of the foregoing. Optional pharmaceutical excipients such as diluents, lubricants, glidants, flavorants, colorants, etc. or a combination comprising at least one of the foregoing may also be included in the compression matrix.

The solid, oral quinine formulations, although using polymeric coating materials known for the preparation of sustained-, extended-, delayed- or pulsed-release formulations, exhibit immediate-release profiles both in vivo and in vitro. An immediate-release formulation is one that has not been modified to provide a release profile that is delayed, extended, sustained, pulsed, or controlled. By “immediate-release” is meant a conventional or non-modified release. As used herein, immediate-release is not controlled-, sustained-, extended-, delayed- or pulsed-release. An immediate-release dosage form may exhibit a release profile as measured in an in vitro dissolution test where greater than or equal to about 75% of the active agent is released within two hours, specifically within one hour after combining the formulation with 900 ml of a dissolution medium, specifically 0.1 N HCl or 0.1 N HCl containing pepsin. In another embodiment, an immediate-release dosage form may exhibit a release profile as measured in an in vitro dissolution test where greater than or equal to about 85% of the active agent is released within 45 minutes after combining the formulation with 900 ml dissolution medium of 0.1 N HCl or 0.1 N HCl containing pepsin (activity of pepsin between 607,500 to 750,000 Units per liter of dissolution medium). Exemplary dissolution conditions include testing according to USP 32 <711>, incorporated herein in its entirety, test method 1 basket at 37° C.±0.5° C., 100 rpm shaft speed.

In one embodiment, the immediate-release dosage form exhibits an immediate-release profile in vivo where the Tₘₐₓ is about 4 hours or less, specifically about 3.5 hours or less, and more specifically about 3 hours or less. The Tₘₐₓ can be determined after administration to a test group of about twenty-five or more healthy humans in the fasted state.

The solid, oral quinine formulation can be described by its pharmacokinetic or dissolution profiles. “Pharmacokinetic parameters” describe the in vivo characteristics of an active agent (or surrogate marker for the active agent) over time, such as plasma concentration (C), Cₘₐₓ, Cₙ, C₂₄, Tₘₐₓ, and AUC. “Cₘₐₓ” is the measured concentration of the active agent in the plasma at the point of maximum concentration. “Cₙ” is the measured concentration of an active agent in the plasma at about n hours after administration. “C₂₄” is the measured concentration of an active agent in the plasma at about 24 hours after administration. The term “Tₘₐₓ” refers to the time at which the measured concentration of an active agent in the plasma is the highest after administration of the active agent. “AUC” is the area under the curve of a graph of the measured concentration of an active agent (typically plasma concentration) vs. time, measured from one time point to another time point. For example AUC₀₋ₜ is the area under the curve of plasma concentration versus time from time 0 to time t. The AUC_{0-∞} or AUC_{0-INF} is the calculated area under the curve of plasma concentration versus time from time 0 to time infinity.

“Bioavailability” means the extent or rate at which an active agent is absorbed into a living system or is made available at the site of physiological activity. For active agents that are intended to be absorbed into the bloodstream, bioavailability data for a given formulation may provide an estimate of the relative fraction of the administered dose that is absorbed into the systemic circulation. “Bioavailability” can be characterized by one or more pharmacokinetic parameters.

In one embodiment, the solid, oral quinine formulation is bioequivalent to a reference drug. In one embodiment, bioequivalence is any definition thereof as promulgated by the U.S. Food and Drug Administration or any successor agency thereof. In a specific embodiment, bioequivalence is determined according to the Federal Drug Administration's (FDA) guidelines and criteria, including “GUIDANCE FOR INDUSTRY BIOAVAILABILITY AND BIOEQUVALENCE STUDIES FOR ORALLY ADMINISTERED DRUG PRODUCTS—GENERAL CONSIDERATIONS” available from the U.S. Department of Health and Human Services (DHHS), Food and Drug Administration (FDA), Center for Drug Evaluation and Research (CDER) March 2003 Revision 1; and “GUIDANCE FOR INDUSTRY STATISTICAL APPROACHES TO ESTABLISHING BIOEQUIVALENCE” DHHS, FDA, CDER, January 2001, both of which are incorporated herein in their entirety.

In another embodiment, bioequivalence is determined according to the European Medicines Agency (EMEA) document “Note for Guidance on the Investigation of Bioavailability and Bioequivalence”, issued Jul. 26, 2001, available from EMEA.

“Reference drug” means the oral quinine sulfate capsule product as described in U.S. Federal Food and Drug Administration's New Drug Application No. 021799 approved on Aug. 12, 2005 (324 mg quinine sulfate) and by its brand name Qualaquin®. Qualaquin® capsules contain 324 mg quinine sulfate ((C₂₀H₂₄N₂O₂)₂.H₂SO₄.2H₂O) powder (269 mg free base), 82 mg corn starch, 40 mg talc, and 4 mg magnesium stearate. Qualaquin® is formulated for immediate-release. Quinine sulfate capsules USP, 324 mg (Qualaquin®) is approved for treatment of uncomplicated Plasmodium falciparum malaria in adults. The recommended quinine dose in adults is 648 mg (two capsules) every 8 hours three times daily for 7 days.

In one embodiment, the quinine formulation is bioequivalent to a reference drug according to New Drug Application No. 021799 when tested in a group of five or more healthy humans in the fasted or fed state.

In an embodiment, bioequivalence of the quinine formulation to a reference drug is determined by an in vivo bioequivalence study to determine a pharmacokinetic parameter for the quinine formulation. Specifically, bioequivalence can be determined by an in vivo bioequivalence study comparing a pharmacokinetic parameter for the two compositions. A pharmacokinetic parameter for the quinine formulation or the reference drug can be measured in a single or multiple dose bioequivalence study using a replicate or a nonreplicate design. For example, the pharmacokinetic parameters for a quinine formulation of the present invention and for a reference drug can be measured in a single dose bioequivalence study using a two-period, two-sequence crossover design. Alternately, a four-period, replicate design crossover study may also be used. Single doses of the test quinine formulation and reference drug are administered and blood or plasma levels of the active agent are measured over time. Pharmacokinetic parameters characterizing rate and extent of active agent absorption are evaluated statistically.

The area under the plasma concentration-time curve from time zero to the time of measurement of the last quantifiable concentration (AUC₀₋ₜ) and to infinity (AUC_{0-∞}), Cₘₐₓ, and Tₘₐₓ can be determined according to standard techniques. Statistical analysis of pharmacokinetic data is performed on logarithmic transformed data (e.g., AUC₀₋ₜ, AUC_{0-∞}, or Cₘₐₓ data) using analysis of variance (ANOVA).

In some embodiments a single dose pharmacokinetic study is performed under non-fasted (“fed”) or fasted conditions. When tested under fed conditions, the formulation is administered with a high fat meal. An exemplary high fat meal includes the test meal disclosed in the document Guidance for Industry, Food-Effect Bioavailability and Fed Bioequivalence Studies, U.S. Department of Health and Human Services Food and Drug Administration, Center for Drug Evaluation and Research (CDER), Center for Biologics Evaluation and Research (CBER) issued December 2002 and available at http://www.fda.gov/cder/guidance/index.htm. The exemplary high-fat meal contains approximately 50 percent of the total caloric content of the meal as fat and contains approximately 800 to 1000 calories; 500-600 calories from fat. As used herein, the term “fat” is used in its conventional, art-recognized meaning.

Under U.S. FDA guidelines, two products (e.g. an inventive composition and Qualaquin®) or methods (e.g., dosing under fed versus fasted conditions) are bioequivalent if the 90% Confidence Interval (CI) limits for a ratio of the geometric mean of logarithmic transformed AUC_{0-∞}, AUC₀₋ₜ, and Cₘₐₓ for the two products or two methods are about 0.80 to about 1.25.

To show bioequivalence between two products or methods pursuant to Europe's EMEA guidelines, the 90% CI limits for a ratio of the geometric mean of logarithmic transformed AUC_{0-∞} and AUC₀₋ₜ for the two products or methods are about 0.80 to about 1.25. The 90% CI limits for a ratio of the geometric mean of logarithmic transformed Cₘₐₓ for the two products or methods can have a wider acceptance range when justified by safety and efficacy considerations. For example the acceptance range can be about 0.70 to about 1.43, specifically about 0.75 to about 1.33, and more specifically about 0.80 to about 1.25.

In one embodiment, in a given experiment, a quinine formulation is considered to be bioequivalent to Qualaquin® if both the Test/Reference ratio for the geometric mean of logarithmic transformed AUC_{0-∞}, AUC₀₋ₜ, or Cₘₐₓ ratio along with its corresponding lower and upper 90% CI limits are within a lower limit of about 0.80 and an upper limit of about 1.25. Thus, for direct comparison between a quinine formulation and Qualaquin®, it is sometimes preferred to determine the pharmacokinetic parameters for the quinine formulation and Qualaquin® side-by side in the same pharmacokinetic study.

In another embodiment, the 90% confidence limits of a ratio of a geometric mean of logarithmic transformed AUC_{0-∞} of the quinine formulation to a geometric mean of logarithmic transformed AUC_{0-∞} of a reference drug according to New Drug Application No. 021799 is about 0.80 to about 1.25 when tested in a group of five or more healthy humans in the fasted or fed state.

In yet another embodiment, the 90% confidence limits of a ratio of a geometric mean of logarithmic transformed AUC₀₋ₜ of the quinine formulation to a geometric mean of logarithmic transformed AUC₀₋ₜ of a reference drug according to New Drug Application No. 021799 is about 0.80 to about 1.25 when tested in a group of five or more healthy humans in the fasted or fed state.

In yet another embodiment, the 90% confidence limits of a ratio of a geometric mean of logarithmic transformed Cₘₐₓ of the quinine formulation to a geometric mean of logarithmic transformed Cₘₐₓ of a reference drug according to New Drug Application No. 021799 is about 0.80 to about 1.25 when tested in a group of five or more healthy humans in the fasted or fed state.

In one embodiment, the formulation is bioequivalent to a reference drug product according to New Drug Application No. 021799 when tested in a group of five or more healthy humans in the fasted or fed state, wherein bioequivalence is determined according to “GUIDANCE FOR INDUSTRY BIOAVAILABILITY AND BIOEQUVALENCE STUDIES FOR ORALLY ADMINISTERED DRUG PRODUCTS—GENERAL CONSIDERATIONS” DHHS, FDA, CDER, March 2003 Revision 1; and “GUIDANCE FOR INDUSTRY STATISTICAL APPROACHES TO ESTABLISHING BIOEQUIVALENCE” DHHS, FDA, CDER, January 2001.

In another embodiment, the quinine formulation when administered under a fed state is bioequivalent to the quinine formulation when administered under a fasted state to five or more healthy humans.

In another embodiment, the 90% confidence limits of a ratio of a geometric mean of logarithmic transformed AUC_{0-∞} of the quinine formulation when tested in a group of five or more healthy humans in the fed state to a geometric mean of logarithmic transformed AUC_{0-∞} of the quinine formulation when tested in a group of five or more healthy humans in the fasted state is about 0.80 to about 1.25.

In one embodiment, the 90% confidence limits of a ratio of a geometric mean of logarithmic transformed AUC₀₋ₜ of the quinine formulation when tested in a group of five or more healthy humans in the fed state to a geometric mean of logarithmic transformed AUC₀₋ₜ of the quinine formulation when tested in a group of five or more healthy humans in the fasted state is about 0.80 to about 1.25.

In another embodiment, the 90% confidence limits of a ratio of a geometric mean of logarithmic transformed Cₘₐₓ of the quinine formulation when tested in a group of five or more healthy humans in the fed state to a geometric mean of logarithmic transformed Cₘₐₓ of the quinine formulation when tested in a group of five or more healthy humans in the fasted state is about 0.80 to about 1.25.

The quinine formulation when tested in a group of five or more healthy humans in the fasted state and in the fed state exhibits a ratio of fed state Cₘₐₓ divided by a fasted state Cₘₐₓ of about 85 to about 125, specifically about 90 to about 120, more specifically about 95 to about 115, and yet more specifically about 100 to about 110.

The quinine formulation when tested in a group of five or more healthy humans in the fasted state and in the fed state exhibits a ratio of fed state AUC₀₋ₜ divided by a fasted state AUC₀₋ₜ of about 85 to about 125, specifically about 90 to about 120, more specifically about 95 to about 115, and yet more specifically about 100 to about 110.

The quinine formulation when tested in a group of five or more healthy humans in the fasted state and in the fed state exhibits a ratio of fed state AUC_{0-∞} divided by a fasted state AUC_{0-∞} of about 85 to about 125, specifically about 90 to about 120, more specifically about 95 to about 115, and yet more specifically about 100 to about 110.

In yet another embodiment, the quinine formulation when tested in a group of five or more healthy humans in the fasted state and in the fed state exhibits a. a ratio of fed state Cₘₐₓ divided by a fasted state Cₘₐₓ; b. a ratio of fed state AUC₀₋ₜ divided by a fasted state AUC₀₋ₜ; or c. a ratio of fed state AUC_{0-∞} divided by a fasted state AUC_{0-∞} of about 95 to about 115.

The release of quinine from the quinine formulations can be described by its dissolution profile. A dissolution profile is a plot of the cumulative amount of active agent released as a function of time. A dissolution profile can be measured utilizing the Drug Release Test <724>, which incorporates standard test USP 32 (Test <711>). A profile is characterized by the test conditions selected such as, for example, apparatus type, shaft speed, temperature, volume, and pH of the dissolution medium. More than one dissolution profile may be measured. For example, a first dissolution profile can be measured at a pH level approximating that of the stomach, and a second dissolution profile can be measured at a pH level approximating that of one point in the intestine or several pH levels approximating multiple points in the intestine.

A highly acidic pH may be employed to simulate the stomach and a less acidic to basic pH may be employed to simulate the intestine. By the term “highly acidic pH” is meant a pH of about 1 to about 4.5. A pH of about 1.2, for example, can be used to simulate the pH of the stomach. By the term “less acidic to basic pH” is meant a pH of greater than about 4 to about 7.5, specifically about 6 to about 7.5. A pH of about 6 to about 7.5, specifically about 6.8, can be used to simulate the pH of the intestine.

The quinine formulation may be tested using a USP Type I apparatus (basket), at 100 rpm, and 900 mL of dissolution media selected from the group of purified water, acidic buffer of pH 4.5, 0.1 N HCl, 0.1 N HCl with added pepsin, and pH 6.8 phosphate buffer.

In one embodiment, the quinine formulation exhibits a dissolution profile that is substantially the same as a dissolution profile of an equivalent strength of a reference drug according to New Drug Application No. 021799 wherein the dissolution profile is determined using the conditions according to USP 32 <711> test method 1 basket, using of 900 ml of 0.1N HCl, optionally containing pepsin (activity of pepsin between 607,500 to 750,000 Units per liter of dissolution medium), at 37° C.±0.5° C., and 100 rpm shaft speed for 90 minutes followed by 250 rpm thereafter. “Substantially the same dissolution profile” means the quinine formulation releases an amount of active agent within about 10% of the amount released from the reference drug according to New Drug Application No. 021799 (Qualaquin®) at any give time point when tested under a dissolution study.

In another embodiment, the quinine formulation exhibits a dissolution profile such that after combining the formulation with 900 ml of 0.1N HCl, optionally containing pepsin (activity of pepsin between 607,500 to 750,000 Units per liter of dissolution medium), at 37° C.±0.5° C. according to USP 32 <711> test method 1 basket, 100 rpm shaft speed, greater than or equal to 85% of the active agent is released within 45 minutes.

In another embodiment, the quinine formulation exhibits a dissolution profile such that after combining the formulation with 900 ml of 0.1N HCl, optionally containing pepsin (activity of pepsin between 607,500 to 750,000 Units per liter of dissolution medium), at 37° C.±0.5° C. according to USP 32 <711> test method 1 basket, 100 rpm shaft speed, about 55 to about 100 wt. %, specifically about 65 to about 100, and yet more specifically about 75 to about 100 wt. % of the total amount of active agent is released within 1 hour.

In another embodiment, the quinine formulation exhibits a dissolution profile such that after combining the formulation with 900 ml of 0.1N HCl, optionally containing pepsin (activity of pepsin between 607,500 to 750,000 Units per liter of dissolution medium), at 37° C.±0.5° C. according to USP 32 <711> test method 1 basket, 100 rpm shaft speed, greater than or equal to 80% of the active agent is released within 60 minutes.

In yet another embodiment, the quinine formulation exhibits a dissolution profile such that after combining the formulation with 900 ml of 0.1N HCl, optionally containing pepsin (activity of pepsin between 607,500 to 750,000 Units per liter of dissolution medium), at 37° C.±0.5° C. according to USP 32 <711> test method 1 basket, 100 rpm shaft speed, greater than or equal to 85% of the active agent is released within 60 minutes.

In yet another embodiment, the quinine formulation exhibits a dissolution profile such that after combining the formulation with 900 ml of 0.1N HCl, optionally containing pepsin (activity of pepsin between 607,500 to 750,000 Units per liter of dissolution medium), at 37° C.±0.5° C. according to USP 32 <711> test method 1 basket, 100 rpm shaft speed, greater than or equal to 90% of the active agent is released within 60 minutes.

In yet another embodiment, the quinine formulation exhibits a dissolution profile such that after combining the formulation with 900 ml of 0.1N HCl, optionally containing pepsin (activity of pepsin between 607,500 to 750,000 Units per liter of dissolution medium), at 37° C.±0.5° C. according to USP 32 <711> test method 1 basket, 100 rpm shaft speed, greater than or equal to 95% of the active agent is released within 60 minutes.

In yet another embodiment, the amount of quinine sulfate released from the formulation at 1.5 hour varies by +/−about 12% from an amount of quinine sulfate released from a reference drug product according to New Drug Application No. 021799 when the formulation and the reference drug product are tested under dissolution conditions according to USP 32 <711> test method 1 basket, using 900 ml of 0.1N HCl, optionally containing pepsin (activity of pepsin between 607,500 to 750,000 Units per liter of dissolution medium), at 37° C.±0.5° C., and 100 rpm shaft speed.

A particular obstacle with a sprinkle formulation is that it is often administered to a patient several minutes after the patient or caregiver has prepared it. Such a time delay can allow for the sprinkle matrix, typically applesauce, to dissolve the sprinkles. By controlling the subunit core size and the particular coatings and amounts, the immediate-release solid, oral quinine formulations have minimum leaching of quinine which allows the sprinkle to be mixed with applesauce matrix for several minutes, sometimes up to an hour prior to administration without exhibiting a bitter taste. Prevention or reduction of leaching results in a more palatable sprinkle formulation and patient acceptability.

The suitability of the quinine formulation for use as a sprinkle formulation on food can be analyzed by a leaching study to determine whether quinine is released into the sprinkle matrix prior to ingestion by the patient.

In one embodiment, the quinine formulation leaches less than 0.6%, specifically less than 0.5%, more specifically less than 0.05%, and yet more specifically less than 0.01% (from a range starting from 0%) quinine as determined by reverse-phase High Performance Liquid Chromatography (HPLC) analysis on a sample taken at 10 minutes from the time the formulation as a sprinkle is mixed with four ounces of unsweetened applesauce, chocolate pudding, or four fluid ounces of orange juice. The test sample of the quinine formulation can comprise 648 mg quinine sulfate. The HPLC analysis can be carried out using a reverse-phase column (e.g., Waters XBridge Shield RP18, 3.5 μm, 3.0×150 mm); at a column temperature of about 30° C.; a flow rate of 0.5 mL/minute; injection volume of 10 μL; detection at 249 nm; and mobile phase of 10 mM Ammonium Bicarbonate Buffer pH 9.5:Acetonitrile:Methanol (650:300:50). Sample preparation for the HPLC analysis includes weighing a five gram aliquot of the applesauce or chocolate pudding ensuring no subunit is included in the aliquot; adding about 30 ml diluent (10 mM Ammonium Bicarbonate Buffer pH 9.5:Acetonitrile:Methanol (650:300:50)); shaking the flask for 15 minutes using a wrist action shaker; adding diluent to result in 50 ml volume; mixing; centrifuging a portion of the prepared sample at 3000 rpm for 15 minutes; and testing the supernatant by reverse-phase HPLC analysis.

In one embodiment, the quinine formulation leaches less than 0.1%, specifically less than 0.05%, more specifically less than 0.01%, and yet more specifically less than 0.001% (from a range starting from 0%) quinine as determined by HPLC analysis on a sample taken at 10 minutes from the time the formulation as a sprinkle is sprinkled onto five grams of unsweetened applesauce. The test sample of the quinine formulation can comprise 648 mg quinine sulfate. The HPLC analysis can be carried out using a reverse-phase column (e.g., Waters XBridge Shield RP18, 3.5 μm, 3.0×150 mm); at a column temperature of about 30° C.; a flow rate of 0.5 mL/minute; injection volume of 10 μL; detection at 249 nm; and mobile phase of 10 mM Ammonium Bicarbonate Buffer pH 9.5:Acetonitrile:Methanol (650:300:50). Sample preparation for the HPLC analysis includes removing the subunits from the applesauce matrix; weighing a 2.5 gram aliquot of the applesauce into a 25 ml volumetric flask ensuring no subunit is included in the aliquot; adding about 15 ml diluent (10 mM Ammonium Bicarbonate Buffer pH 9.5:Acetonitrile:Methanol (650:300:50)); shaking the flask for 15 minutes using a wrist action shaker; adding diluent to volume; mixing; centrifuging a portion of the prepared sample at 3000 or 15,000 rpm for 15 minutes; and testing the supernatant by reverse-phase HPLC analysis.

The suitability of the quinine formulation for use as a sprinkle formulation on food can also be analyzed by conducting a palatability study using a visual analog scale (VAS). In one embodiment, subjects are provided a sprinkle quinine formulation on a food vehicle (e.g. applesauce, pudding, juice, and the like) and asked to gauge the palatability of the dosage using a horizontally oriented, uniformly spaced sequence of integers from “0” to “10” on a 10 cm visual analog scale (VAS). Palatability is measured by how difficult it is to take the dosage form (e.g. to complete a 7-day treatment TID), where the lower end is “Not at all difficult” (0) and the upper end is “Extremely difficult” (10). The sprinkle form can be mixed with the food vehicle and administered to the subjects at various time points from immediately to a period of time after the sprinkle and food vehicle are combined (e.g. 10, 15, 30, 60 minutes, etc.). In one embodiment, a quinine sprinkle formulation described herein is analyzed according to the described palatability study and results in a mean VAS score of 3 or less when the time to administration is 0, 10, 15, 30, or 60 minutes after the sprinkle and food vehicle are combined.

Quinine sulfate exhibits pH dependent solubility in aqueous media. Accordingly, it would be beneficial to prepare microparticles or nanoparticles of quinine sulfate as a way of increasing its solubility, and perhaps increase its in vivo bioavailability. Accordingly, the preparation of microparticles and nanoparticles of quinine sulfate is provided herein.

In one embodiment, quinine, specifically quinine sulfate, is micronized using techniques known in the art to provide quinine having an average diameter of about 1 to about 500 micrometers, specifically about 5 to about 250 micrometers, and more specifically about 25 to about 100 micrometers.

Any conventional means of measuring particle size can be used, for example laser light scattering techniques.

In another embodiment, quinine, specifically quinine sulfate, is micronized using techniques known in the art to provide quinine having a particle size distribution D(v,0.9) of less than 10 micrometers as measured by laser diffraction particle size analysis, specifically about 2 to about 9, more specifically about 3 to about 8, and yet more specifically about 4 to about 7 micrometers.

In another embodiment, quinine, specifically quinine sulfate, is micronized using techniques known in the art to provide quinine having a particle size distribution D(v,0.5) of less than 5 micrometers as measured by laser diffraction particle size analysis, specifically about 0.1 to about 4, more specifically about 0.5 to about 3, and yet more specifically about 1 to about 2 micrometers.

In another embodiment, quinine, specifically quinine sulfate, is micronized using techniques known in the art to provide quinine having a particle size distribution D(v,0.1) of less than 2 micrometers as measured by laser diffraction particle size analysis, specifically about 0.1 to about 1, more specifically about 0.3 to about 0.9, and yet more specifically about 0.5 to about 0.8 micrometers.

In one embodiment, the quinine, specifically quinine sulfate, is micronized using a jet mill micronizer optionally in the presence of a surfactant.

Exemplary surfactants include amphoteric, non-ionic, cationic or anionic surfactants. Particular examples include sodium lauryl sulfate, monooleate, monolaurate, monopalmitate, monostearate or another ester of polyoxyethylene sorbitane, sodium dioctylsulfosuccinate, lecithin, stearylic alcohol, cetostearylic alcohol, cholesterol, polyoxyethylene ricin oil, polyoxyethylene fatty acid glycerides, Poloxamer®, or a combination comprising at least one of the forgoing surfactants.

The solid, oral quinine formulations disclosed herein can be used to treat a patient in need of quinine therapy. In one embodiment, a method of treating involves administering the quinine formulation for the treatment of sp. Falciparum infection, uncomplicated Plasmodium falciparum malaria, severe or complicated Plasmodium falciparum malaria, treatment of Plasmodium vivax infection, treatment of babesiosis caused by Babesia microti, the prevention of malaria, or the treatment or prevention of leg cramps (e.g., nocturnal).

In one embodiment, a method of administering quinine comprises administering two unit dosage forms of a quinine formulation TID to a patient in need of quinine therapy, wherein the quinine formulation comprises a solid oral dosage form comprising a plurality of coated subunits, wherein each coated subunit comprises a core subunit comprising quinine or a pharmaceutically acceptable salt thereof, and a pharmaceutically acceptable excipient, and a coating on the outside of the core subunit, wherein the coating comprises a polymeric coating material, wherein the polymeric coating material is chitosan; ethylcellulose; hydroxypropyl methylcellulose acetate succinate; cellulose acetate phthalate; a (meth)acrylic acid copolymer; hydroxypropyl methylcellulose succinate; cellulose acetate succinate; cellulose acetate hexahydrophthalate; hydroxypropyl methylcellulose hexahydrophthalate; hydroxypropyl methylcellulose phthalate; cellulose propionate phthalate; cellulose acetate maleate; cellulose acetate trimellitate; cellulose acetate butyrate; cellulose acetate propionate; a polyvinylacetate phthalate; zein; or a combination thereof; optionally in combination with a plasticizer, a stabilizer, a water-soluble component, an anti-tacking agent, a surfactant, or a combination thereof; wherein the quinine formulation exhibits immediate-release profile; and wherein the quinine formulation can be administered as a single unit solid oral dosage form or administered as a sprinkle on food.

Also included herein are pharmaceutical products (kits) useful, for example, for the treatment or prevention of parasitic diseases caused by Plasmodium species (e.g. sp. Plasmodium, Plasmodium falciparum, etc.), the treatment and prophylaxis of leg cramps, or the treatment of babesiosis caused by Babesia microti, which comprise one or more containers containing a quinine formulation as disclosed herein and optionally information or published material, e.g as product inserts or product labels. The information can indicate quantities of the components to be administered, guidelines for administration, safety issues, and the like.

The kits may further comprise one or more conventional pharmaceutical kit components, such as, for example, one or more containers to aid in facilitating compliance with a particular dosage regimen; one or more carriers; etc. Exemplary kits can be in the form of bubble or blister pack cards, optionally arranged in a desired order for a particular dosing regimen. Suitable blister packs that can be arranged in a variety of configurations to accommodate a particular dosing regimen are well known in the art or easily ascertained by one of ordinary skill in the art.

The quinine formulations can be administered without regard to food. Thus, the quinine formulations can be administered with or without food. It has been found that the quinine formulations improve patient compliance, since it can be taken with or without food. Furthermore, the quinine formulation reduces or eliminates the incidence of gastric irritation and upset that can occur with the administration of powdered quinine sulfate in capsule form in the absence of food. The reduction or elimination of gastric irritation and upset incidents is likely due to the reduced amount of quinine present at the surface of the formulation, which in turn reduces or eliminates the likelihood the patient will experience a bitter taste.

In another embodiment, a method of reducing or eliminating incidents of gastric upset and irritation experienced by the administration of capsule formulations of powdered quinine without food comprises administering a quinine formulation without food, wherein the quinine formulation comprises a solid oral dosage form comprising a plurality of coated subunits, wherein each coated subunit comprises a core subunit comprising quinine or a pharmaceutically acceptable salt thereof, and a pharmaceutically acceptable excipient, and a coating on the outside of the core subunit, wherein the coating comprises a polymeric coating material, wherein the polymeric coating material is chitosan; ethylcellulose; hydroxypropyl methylcellulose acetate succinate; cellulose acetate phthalate; a (meth)acrylic acid copolymer; hydroxypropyl methylcellulose succinate; cellulose acetate succinate; cellulose acetate hexahydrophthalate; hydroxypropyl methylcellulose hexahydrophthalate; hydroxypropyl methylcellulose phthalate; cellulose propionate phthalate; cellulose acetate maleate; cellulose acetate trimellitate; cellulose acetate butyrate; cellulose acetate propionate; a polyvinylacetate phthalate; zein; or a combination thereof; optionally in combination with a plasticizer, a stabilizer, a water-soluble component, an anti-tacking agent, a surfactant, or a combination thereof; and wherein the quinine formulation exhibits immediate-release profile.

In one embodiment, an oral quinine formulation comprises a solid oral dosage form comprising a plurality of taste-masked coated subunits, wherein each coated subunit comprises a core subunit comprising quinine sulfate and a pharmaceutically acceptable excipient, and a coating on the outside of the core subunit, wherein the coating is a. about 1 to about 7%, specifically about 2 to about 6%, and more specifically about 3 to about 5% weight gain based on the weight of the core subunit of a coating consisting essentially of a combination of ethylcellulose and hydroxypropyl methylcellulose, and optionally a plasticizer, stabilizer, an anti-tacking agent, a surfactant, or a combination thereof, wherein the ethylcellulose and hydroxypropyl methylcellulose are in a weight ratio of about 2:1 to about 1:2, specifically about 1.8:1 to about 1:1.8, more specifically about 1.5:1 to about 1:1.5, more specifically about 1.2:1 to about 1:1.2, and still yet more specifically about 1:1; or b. about 6 to about 14%, specifically about 8 to about 12%, and more specifically about 9 to about 11% weight gain based on the weight of the core subunit of a coating consisting essentially of a combination of cellulose acetate phthalate and hydroxypropyl methylcellulose, and optionally a plasticizer, wherein the cellulose acetate phthalate and hydroxypropyl methylcellulose are in a weight ratio of about 3:1 to about 1:1, specifically about 2.6:1 to about 2:1, and more specifically about 2.5:1 to about 2.1:1; wherein the quinine formulation exhibits immediate-release profile, and wherein the quinine formulation can be administered as a single unit solid oral dosage form or administered as a sprinkle on food.

The following examples further illustrate the invention but, of course, should not be construed as in any way limiting its scope.

### EXAMPLES

### Example 1

### Preparation of Quinine Sulfate Core Minitablets

Quinine sulfate core minitablets are prepared by a compression process using the components provided in Table 1 below.

TABLE 1

| | Core Formulation | | |
|---|---|---|---|
| | 1 | 2 | 3 |
| Component | Mg/minitablet | | |
| Base granules | | | |
| Quinine sulfate 2:1 dihydrate | 8.10 | 8.10 | 8.10 |
| ((C₂₀H₂₄N₂O₂)₂•H₂SO₄•2H₂O) | | | |
| Carnauba wax | 0.45 | 0.45 | 0.45 |
| Hydroxy Propylcellulose (Klucel | 0.13 | — | — |
| HXF) | | | |
| Stearic acid | 0.10 | 0.10 | 0.10 |
| SD3A Alcohol (L) | * | * | * |
| Core granules | | | |
| Base granules | 8.78 | 8.65 | 8.65 |
| Hydroxy Propylcellulose (Klucel | — | 0.13 | 0.13 |
| HXF) | | | |
| Microcrystaline cellulose (Avicel | 0.97 | 0.97 | 0.94 |
| pH101) | | | |
| Croscarmellose Sodium (Ac-Di-Sol) | 0.03 | 0.03 | 0.03 |
| Silicon Dioxide (Syloid 244 FP) | 0.12 | 0.12 | 0.12 |
| Magnesium stearate | 0.10 | 0.10 | 0.13 |
| Total | 10.00 | 10.00 | 10.00 |

* Not present in final formulation

The core minitablets are prepared by wet granulating quinine sulfate, carnauba wax, hydroxyl propylcellulose, and stearic acid in alcohol to form granules. The granules are dried and then blended with microcrystalline cellulose, croscarmellose sodium, silicon dioxide, and optionally a second portion of hydroxyl propylcellullose. Magnesium stearate is added at the end of the mixing process to form a core granulate mixture. The core granulate mixture is compressed into cylinder-shaped minitablets 0.08 inches (−2 mm) thick (dome to dome) and 2.5 mm long.

### Example 2

### Preparation of Coated Quinine Sulfate Minitablets

Coated quinine sulfate minitablets are prepared by coating core minitablets with a coating to provide taste-masking. The coating minitablet formulations are provided in Table 2 below.

TABLE 2

| | Mg/minitablet | | | | | | | | |
|---|---|---|---|---|---|---|---|---|---|
| | Formulation (% wt gain) | | | | | | | | |
| | 2A | 2B | 2C | 2D | 2E \hat{ } | 2F | 2G | 2H | 2I |
| Component | (4%) | (4%) | (10%) | (20%) | (6%) | (6%) | (20%) | (6%) | (12%) |
| Core from | 1 | 1 | 1 | 1 | 2 | 2 | 2 | 3 | 3 |
| Example 1 | | | | | | | | | |
| Intermediate | | | | | | | | | |
| coating | | | | | | | | | |
| Core minitablet | 10.0 | 10.0 | 10.0 | 10.0 | 10.0 | 10.0 | 10.0 | 10.0 | 10.0 |
| Opadry clear | 0.30 | 0.30 | 0.30 | 0.30 | 0.30 | 0.30 | 0.30 | 0.30 | 0.30 |
| (YS-3-7011; | | | | | | | | | |
| hydroxypropyl | | | | | | | | | |
| methylcellulose- | | | | | | | | | |
| based coating) | | | | | | | | | |
| Purified water * | * | * | * | * | * | * | * | * | * |
| Taste-masking | | | | | | | | | |
| coating | | | | | | | | | |
| Intermediate | 10.3 | 10.3 | 10.3 | 10.3 | 10.3 | 10.3 | 10.3 | 10.3 | 10.3 |
| coated | | | | | | | | | |
| minitablet | | | | | | | | | |
| Surelease | 0.206 | — | — | — | — | — | — | — | — |
| Opadry clear | 0.206 | — | — | — | — | — | — | — | — |
| (YS-3-7011) | | | | | | | | | |
| EUDRAGIT RS | — | 0.09 | — | — | — | — | — | — | — |
| EUDRAGIT | — | 0.28 | — | — | — | — | — | — | — |
| RL | | | | | | | | | |
| Cellulose | — | — | 0.61 | 1.22 | — | — | — | — | — |
| acetate | | | | | | | | | |
| phthalate | | | | | | | | | |
| aqueous | | | | | | | | | |
| dispersion | | | | | | | | | |
| (Aquacoat CPD | | | | | | | | | |
| 30D) | | | | | | | | | |
| Hydroxypropyl | — | — | 0.26 | 0.52 | — | — | — | — | — |
| methylcellulose | | | | | | | | | |
| (Methocel E5) | | | | | | | | | |
| EUDRAGIT | — | — | — | — | 0.66 | 0.66 | 2.57 | — | — |
| EPO | | | | | | | | | |
| EUDRAGIT | — | — | — | — | — | — | — | 0.59 | 1.27 |
| L30D55 | | | | | | | | | |
| Triethyl citrate | — | 0.04 | 0.16 | 0.32 | — | — | — | 0.06 | 0.14 |
| Talc | — | 0.21 | — | — | 0.22 | 0.22 | 0.86 | 0.36 | 0.76 |
| Sodium lauryl | — | — | — | — | 0.07 | 0.07 | 0.26 | — | — |
| sulfate | | | | | | | | | |
| Stearic acid | — | — | — | — | 0.10 | 0.10 | 0.38 | — | — |
| Purified water * | * | — | * | * | * | * | * | * | * |
| SD3A Alcohol | — | * | — | — | — | — | — | — | — |
| (L) * | | | | | | | | | |
| Total | 10.71 | 10.92 | 11.33 | 12.36 | 11.35 | 11.35 | 14.37 | 11.31 | 12.47 |

* Not present in final formulation
\hat{ } Micronized quinine sulfate

The core minitablets are first coated with a mixture of Opadry clear and water and then coated with a taste-masking coating mixture as outlined in Table 2 using a fluid bed coater (e.g. Glatt GPCG-120 with Wurster coater insert) and dried to result in the coated quinine sulfate minitablets.

Coated quinine sulfate minitablets of Formulation 2E contain micronized quinine sulfate as described below in Example 13.

### Example 3

### Preparation of Quinine Sulfate Capsules Containing Coated Minitablets

Immediate-release quinine sulfate capsules are prepared by encapsulating the coated minitablets of Example 2 in hard gelatin capsules to achieve a total of 324 mg of quinine sulfate per capsule (Table 3).

TABLE 3

| | Capsule formulation | | | | | | | | |
|---|---|---|---|---|---|---|---|---|---|
| | 3A | 3B | 3C | 3D | 3E | 3F | 3G | 3H | 3I |
| Coated | 2A | 2B | 2C | 2D | 2E | 2F | 2G | 2H | 2I |
| minitablet of | | | | | | | | | |
| Example 2 | | | | | | | | | |
| Mg coated | 428.4 | 436.8 | 453.2 | 494.4 | 454.0 | 454.0 | 574.8 | 452.4 | 498.8 |
| minitablets/ | | | | | | | | | |
| capsule | | | | | | | | | |

### Example 4

### Dissolution Studies

The capsule formulations of Example 3 containing 324 mg of quinine sulfate are tested for dissolution according to USP <711> using the following equipment and conditions

### UV/VIS Spectrophotometer

0.2-cm UV-Cell

Fixed wavelength about 420 nm and 346 nm

Maximum about 346 nm

Dissolution medium 0.1 N HCl with pepsin
Activity of pepsin between 607,500 to 750,000 Units per liter of dissolution medium
Volume dissolution medium 900 ml
USP Apparatus 1 (basket)

### Speed 100 rpm

Increase speed to 200/250 rpm after 90 minutes

### Temperature 37.0° C.±0.5° C.

Time points: As indicated in tables below
Calculation of weight of pepsin required for 1000 ml dissolution medium

$$Weight of pepsin equivalent to 675 , 000 Units of activity ( g ) = \frac{675 , 000 Units}{A} \times \frac{1 g}{1000 mg}$$

A=activity of pepsin specified on the product label in Units/mg of solid. The results of the dissolution study are provided in Tables 4 to 9 below as weight percent of quinine sulfate released.
“X”-month(s) ACC means (x=1, 3, or 6-months) of accelerated stability study where the dosage form is exposed to conditions of 75° C. and 40 percent relative humidity for the indicated time period.
““X”-month LT” means long term aged dosage forms for the indicated time period at ambient conditions.

TABLE 4

| | | | 3C; | 3C; | 3C; |
|---|---|---|---|---|---|
| | | | CAP/HPMC | CAP/HPMC | CAP/HPMC |
| | | 3C; | (3-month | (6-month | (6-month |
| | | CAP/ | ACC) | LT) | ACC) 0.1N |
| Time | | HPMC | 0.1N HCl | 0.1N HCl w/ | HCl w/ |
| (min) | Qualaquin ® | (T = 0) | w/pepsin | pepsin | pepsin |
| 0 | 0 | 0 | 0 | 0 | 0 |
| 15 | 87 | 73 | 14 | 57 | 16 |
| 30 | 101 | 102 | 69 | 95 | 64 |
| 45 | 101 | 106 | 92 | 103 | 88 |
| 60 | 101 | 107 | 102 | 104 | 100 |
| 90 | — | 107 | 104 | 104 | 104 |
| 120 | — | 107 | 104 | 104 | 104 |

TABLE 5

| | | | 3F; EPO (T = 6- |
|---|---|---|---|
| | | 3F; EPO | month ACC) |
| Time (min) | Qualaquin ® | (T = 0) | pepsin |
| 0 | 0 | 0 | 0 |
| 15 | 87 | 94 | 82 |
| 30 | 101 | 100 | 97 |
| 45 | 101 | 98 | 97 |
| 60 | 101 | 99 | 98 |
| 90 | — | — | 98 |

TABLE 6

| | | | | 3A; | 3A; | 3A; |
|---|---|---|---|---|---|---|
| | | | 3A; | Surelease/ | Surelease/ | Surelease/ |
| | | 3A; | Surelease/ | Opadry | Opadry | Opadry |
| | | Surelease/ | Opadry | (3-month | (6-month | (6-month |
| | | Opadry | (T = 0) | ACC) | LT) 0.1N | ACC) |
| Time | | (T = 0) | 0.1N HCl | 0.1N HCl | HCl | 0.1N HCl |
| (min) | Qualaquin ® | 0.1N HCl | w/pepsin | w/pepsin | w/pepsin | w/pepsin |
| 0 | 0 | 0 | 0 | 0 | 0 | 0 |
| 15 | 87 | 93 | 69 | 69 | 63 | 63 |
| 30 | 101 | 99 | 93 | 93 | 95 | 90 |
| 45 | 101 | 99 | 98 | 98 | 99 | 97 |
| 60 | 101 | 99 | 99 | 99 | 100 | 98 |
| 90 | — | — | 99 | 99 | 100 | 99 |
| 120 | — | — | 99 | 99 | 100 | 99 |

TABLE 7

| | | | 3E; (micronized) |
|---|---|---|---|
| Time | | 3E; (micronized) | (T = 0) 0.1N HCl |
| (min) | Qualaquin ® | (T = 0) 0.1N HCl | w/ pepsin |
| 0 | 0 | 0 | 0 |
| 15 | 87 | 96 | 73 |
| 30 | 101 | 101 | 100 |
| 45 | 101 | 102 | 101 |
| 60 | 101 | 102 | 101 |

TABLE 8

| | | | 3D; CAP/ | 3D; CAP/ | 3D; CAP/ | 3D; CAP/ |
|---|---|---|---|---|---|---|
| | | | HPMC | HPMC | HPMC | HPMC |
| | | 3D; CAP/ | (1-month | (3-month | (6-month | (6-month |
| | | HPMC | ACC) | ACC) | LT) | ACC) |
| Time | | (T = 0) | 0.1N HCl | 0.1N HCl | 0.1N HCl | 0.1N HCl |
| (min) | Qualaquin ® | 0.1N HCl | w/pepsin | w/pepsin | w/ pepsin | w/pepsin |
| 0 | 0 | 0 | 0 | 0 | 0 | 0 |
| 15 | 87 | 78 | 6 | 2 | 8 | 3 |
| 30 | 101 | 94 | 51 | 31 | 64 | 29 |
| 45 | 101 | 100 | 79 | 64 | 87 | 59 |
| 60 | 101 | 101 | 92 | 85 | 96 | 81 |
| 90 | — | 101 | 99 | 98 | 99 | 98 |
| 120 | — | — | 99 | 99 | 99 | 100 |

### Example 5a

### Leaching Studies

A leaching study for the coated minitablets of the Example 3 capsule formulations is performed to determine the amount of quinine sulfate that leaches into applesauce. The amount of leaching correlates to the degree of bitterness that will be experienced when the applesauce containing the minitablets is consumed. The leaching study is performed with two different volumes (4 ounces and 5 grams) of applesauce (Musselman's Naturally Fat Free Applesauce, unsweetened) at six time points. A separate dosing formulation is prepared for each time point.

Dosing formulations with 4 ounces of applesauce is prepared by mixing applesauce with the contents of two 324 mg quinine sulfate capsules containing coated minitablets. Dosing formulations with one teaspoon (5 grams) of applesauce is prepared by the sprinkling the contents of two 324 mg quinine sulfate capsules containing coated minitablets onto the applesauce. At the predetermined time points (5, 10, 15, 30, 45, and 60 minutes), an aliquot of applesauce is taken from the formulation, prepared according to the procedure below, and analyzed by Reverse-Phase High Performance Liquid Chromatography according to the method parameters in Table 10.

TABLE 10

| Parameter | Description |
|---|---|
| Analytical Column | Waters XBridge Shield RP18, 3.5 μm, |
| | 3.0 × 150 mm |
| Column Temperature | 30° C. |
| Autosampler Temperature | Ambient |
| Mobile Phase | 10 mM Ammonium Bicarbonate |
| | Buffer pH 9.5:Acetonitrile:Methanol |
| | (650:300:50) |
| Flow Rate | 0.5 mL/minute |
| Injection Volume | 10 μL |
| Detection | 249 nm |
| Run Time | 17 minutes |
| Standard | Quinine sulfate dihydrate USP; |
| | 0.01 mg/ml in 10 mM Ammonium |
| | Bicarbonate Buffer pH |
| | 9.5:Acetonitrile:Methanol (650:300:50) |

Sample preparation-4 oz: Weigh 4 oz sample of applesauce and sprinkle the contents of two 324 mg quinine sulfate capsules containing coated minitablets onto the top; stir with a plastic spoon. At the predetermined time points, weigh a five gram aliquot into a 50 ml volumetric flask ensuring no minitablet is included in the aliquot. Add about 30 ml diluent (10 mM Ammonium Bicarbonate Buffer pH 9.5:Acetonitrile:Methanol (650:300:50)) and shake flask for 15 minutes using a wrist action shaker. Allow contents to settle. Dilute flask to volume using diluent. Mix well. Centrifuge a portion at 3000 rpm for 15 minutes. Test supernatant by HPLC analysis.

Sample preparation-5 gram: Weigh 5 grams of applesauce onto a plastic teaspoon. Sprinkle contents of two 324 mg quinine sulfate capsules containing coated minitablets onto the applesauce. At the predetermined time points remove the minitablets with the aid of tweezers. Weigh a 2.5 gram aliquot of applesauce into a 25 ml volumetric flask. Add about 15 ml diluent and shake flask for 15 minutes using a wrist action shaker. Allow contents to settle. Dilute flask to volume using diluent. Mix well. Centrifuge a portion at 3000 rpm for 15 minutes. Test supernatant by HPLC analysis.

Reverse-phase HPLC analysis. Quantitation is based on the average combined peak area response of quinine sulfate and dihydroquinine in all injections of Standard Solution 1 made throughout the analytical run.

Determination of quinine sulfate concentration on an anhydrous basis in Standard Solutions:

$${C}_{STD} = \frac{{W}_{STK}}{100} \times DF \times \frac{100 \% - Water content ( \% )}{100 \%} \times {P}_{STD} \times \frac{1000 micrograms}{1 mg}$$

### Where:

CsTD=concentration of quinine sulfate in standard (microgram/ml)
W_{STK}=weight of quinine sulfate dihydate (mg)
100=dilution volume (ml)
DF=dilution factor
P_{STD}=purity of quinine sulfate standard in decimal form

Determination of quinine sulfate content of the sample as mg/formulation of quinine sulfate dihydrate:

$$mg = \frac{{A}_{SPL}}{{A}_{STD}} \times {C}_{STD} \times \frac{Sample volume ( ml )}{Spl ( g )} \times \frac{WUM ( g )}{1000 mg} \times \frac{782.94}{746.91}$$

### Where:

C_{STD}=concentration of quinine sulfate in working standard 1 (microgram/ml)
A_{STD}=averaged combined quinine sulfate and dihydroquinine peak areas in all injections of working standard 1
A_{SPL}=combined quinine sulfate and dihydroquinine peak areas in sample
Spl=actual sample weight (g)
WUM=weight of unfortified matrix (g)
746.91 and 782.94=molecular weights of quinine sulfate anhydrous and quinine sulfate dihydrate, respectively
1000=conversion to mg

The results of the leaching study are provided in Tables 11-14 below.

TABLE 11
CAP/HPMC 10% and CAP/HPMC 20% in 4 oz study

| | Formulation | | | |
|---|---|---|---|---|
| Time Point | 3C; CAP/HPMC 10% | | 3D; CAP/HPMC 20% | |
| (minutes) | mg/4 oz | % of Dose | mg/4 oz | % of Dose |
| 5 | 0.64189 | 0.099 | Not Detected | N/A |
| 10 | 2.8865 | 0.45 | 0.053166 | 0.0082 |
| 15 | 3.1844 | 0.49 | 0.28349 | 0.044 |
| 30 | 5.5562 | 0.86 | 1.3830 | 0.21 |
| 45 | 4.3708 | 0.67 | 1.8989 | 0.29 |
| 60 | 60.982* | 9.4* | 15.179 | 2.3 |

*Sample was diluted

TABLE 12
Eudragit EPO and Surelease/Opadry in 4 oz study

| | Formulation | | | |
|---|---|---|---|---|
| Time Point | 3F; Eudragit EPO | | 3A; Surelease/Opadry | |
| (minutes) | mg/4 oz | % of Dose | mg/4 oz | % of Dose |
| 5 | Not Detected | N/A | 1.4153 | 0.22 |
| 10 | 0.26495 | 0.041 | 2.8174 | 0.43 |
| 15 | 3.5071 | 0.54 | 7.0676 | 1.1 |
| 30 | 81.063* | 13 | 10.565 | 1.6 |
| 45 | 144.13* | 22 | 35.276* | 5.4 |
| 60 | 716.98* | 111** | 27.593* | 4.3 |

*Samples were diluted
**High result was most likely due to the formulation not being homogeneous.

TABLE 13
CAP/HPMC 10% and CAP/HPMC 20% in 5 grams

| | Formulation | | | |
|---|---|---|---|---|
| Time Point | 3C; CAP/HPMC 10% | | 3D; CAP/HPMC 20% | |
| (minutes) | mg/5 grams | % of Dose | mg/5 grams | % of Dose |
| 5 | 0.0042979 | 0.00066 | 0.0033732 | 0.00052 |
| 10 | 0.058255 | 0.0090 | 0.0067569 | 0.0010 |
| 15 | 0.17401 | 0.027 | 0.0073994 | 0.0011 |
| 30 | 0.78226* | 0.12 | 0.11337 | 0.017 |
| 45 | 1.3349* | 0.21 | 0.73792 | 0.11 |
| 60 | 1.8623* | 0.29 | 0.91935* | 0.14 |

*Samples were diluted

TABLE 14
Eudragit EPO and Surelease/Opadry in 5 grams

| | Formulation | | | |
|---|---|---|---|---|
| Time Point | 3F; Eudragit EPO | | 3A; Surelease/Opadry | |
| (minutes) | mg/5 grams | % of Dose | mg/5 grams | % of Dose |
| 5 | Not Detected | N/A | 0.075690 | 0.012 |
| 10 | 0.0045508 | 0.00070 | 0.23935 | 0.037 |
| 15 | 0.0069862 | 0.0011 | 0.36695 | 0.057 |
| 30 | 0.30764 | 0.047 | 0.44942 | 0.069 |
| 45 | 0.68239 | 0.11 | 1.4719* | 0.23 |
| 60 | 1.0444* | 0.16 | 1.7430* | 0.27 |

*Samples were diluted

As shown by the results in Tables 11-14, the formulations of Example 3 provide adequate prevention of leaching of quinine for several minutes.

Formulations 3A and 3C are subject to a leaching study in 4 oz applesauce as previously described. These two formulations are further subject of a leaching study using pulp free orange juice 4 fluid oz (Florida's Natural Orange Juice, Original, No Pulp) and chocolate pudding 3.5 oz (ConAgra Foods Snack Pack Chocolate Pudding). The leaching timepoints are 5 minute, 10 minute, 15 minute, 30 minute, 45 minute, 1 hour, 2 hour, 4 hour, and 8 hour.

For each individual applesauce and chocolate pudding experiment, the vehicle is transferred to a suitable container to obtain an accurate weight of the vehicle. Two 324 mg quinine sulfate capsules of the test formulation is emptied into each vehicle and briefly stirred in with a plastic spoon and stored at room temperature. At the predetermined time points, the test mixture is stirred briefly and a 5 gram aliquot is withdrawn using a disposable transfer pipette, making sure that no minitablets are included in the withdrawn aliquot. All samples are prepared and analyzed as described above, except centrifuging is performed at 15,000 rpm instead of 3000 rpm and the sample run time is 20 minutes.

For each individual orange juice experiment, four fluid ounces of orange juice is poured into a tared 250-mL container to obtain an accurate weight of the vehicle. Two 324 mg quinine sulfate capsules of the test formulation is emptied into the orange juice and briefly stirred in with a plastic spoon and stored at room temperature. At the predetermined time points, the test mixture is stirred briefly and a 5 gram aliquot is withdrawn using a disposable transfer pipette, making sure that no minitablets are included in the withdrawn aliquot. All samples are prepared and analyzed according to the following parameters in Table 15.

TABLE 15

| Parameter | Description | | |
|---|---|---|---|
| Analytical Column | Waters XBridge Phenyl, 3.5 μm, 4.6 × 150 mm | | |
| Column Temperature | 35° C. | | |
| Autosampler | Ambient | | |
| Temperature | | | |
| Mobile Phase A | 50 mM Ammonium Acetate Buffer | | |
| Mobile Phase B | Acetonitrile | | |
| Flow Rate | 1.0 mL/minute | | |
| | Time (minutes) | % A | % B |
| Gradient | 0 | 80 | 20 |
| | 17.0 | 80 | 20 |
| | 17.1 | 30 | 70 |
| | 22.0 | 30 | 70 |
| | 22.1 | 80 | 20 |
| | 35.0 | 80 | 20 |

| | |
|---|---|
| Injection Volume | 15 μL |
| Detection | 249 nm |
| Run Time | 35 minutes |
| Acquisition Time | 18 minutes |

The results are provided below:

TABLE 16
Applesauce

| | | CAP/HPMC 10% | | Surelease/Opadry | |
|---|---|---|---|---|---|
| | | | % Quinine | | % Quinine |
| Time | | | Sulfate | | Sulfate |
| Point | | mg/Cup | Leached | mg/Cup | Leached |
| 5 | minutes | 0.34002 | 0.052 | 0.72762 | 0.11 |
| 10 | minutes | 0.65897 | 0.10 | 2.7543 | 0.43 |
| 15 | minutes | 2.0291 | 0.31 | 1.7090 | 0.26 |
| 30 | minutes | 13.386 | 2.1 | 2.8759 | 0.44 |
| 45 | minutes | 15.021 | 2.3 | 35.726* | 5.5 |
| 1 | hour | 37.658* | 5.8 | 30.104* | 4.6 |
| 2 | hours | 36.023* | 5.6 | 35.759* | 5.5 |
| 4 | hours | 65.489* | 10 | 50.817* | 7.8 |
| 8 | hours | 123.66* | 19 | 210.50* | 32 |

TABLE 17
Chocolate Pudding

| | | CAP/HPMC 10% | | Surelease/Opadry | |
|---|---|---|---|---|---|
| | | | % Quinine | | % Quinine |
| Time | | | Sulfate | | Sulfate |
| Point | | mg/Cup | Leached | mg/Cup | Leached |
| 5 | minutes | < LOQ | N/A | 0.32598 | 0.050 |
| 10 | minutes | 0.15623 | 0.024 | 0.36443 | 0.056 |
| 15 | minutes | 0.41087 | 0.063 | 0.62529 | 0.096 |
| 30 | minutes | 0.67027 | 0.10 | 1.3861 | 0.21 |
| 45 | minutes | 4.4603 | 0.69 | 0.89824 | 0.14 |
| 1 | hour | 3.0847 | 0.48 | 3.0008 | 0.46 |
| 2 | hours | 64.062* | 9.9 | 1.3287 | 0.21 |
| 4 | hours | 36.302* | 5.6 | 17.439 | 2.7 |
| 8 | hours | 47.622* | 7.3 | 64.137* | 9.9 |

N/A not applicable

TABLE 18
Orange Juice

| | | CAP/HPMC 10% | | Surelease/Opadry | |
|---|---|---|---|---|---|
| | | | % Quinine | | % Quinine |
| Time | | | Sulfate | | Sulfate |
| Point | | mg/4 fl oz | Leached | mg/4 fl oz | Leached |
| 5 | minutes | ND | N/A | 0.99745 | 0.15 |
| 10 | minutes | 0.92570 | 0.14 | 7.7021 | 1.2 |
| 15 | minutes | 10.376 | 1.6 | 30.857 | 4.8 |
| 30 | minutes | 20.000 | 3.1 | 77.691 | 12 |
| 45 | minutes | 47.136 | 7.3 | 103.62 | 16 |
| 1 | hour | 58.716 | 9.1 | 124.35 | 19 |
| 2 | hours | 70.294 | 11 | 138.13* | 21 |
| 4 | hours | 191.14* | 29 | 109.75 | 17 |
| 8 | hours | 193.02* | 30 | 271.32* | 42 |

ND—Not Detected
N/A Not Applicable

As indicated by the results in Tables 16-18, the coatings prevented leaching (less than 12%) within thirty minutes, which provides suitable taste masking for a tolerable taste profile in a variety of foods of varying pH (apple sauce 4 or less, orange juice 3-4, and chocolate pudding 5.5-6.5). In applesauce, the formulations provided suitable taste masking for a tolerable taste profile for up to several hours.

An in-use stability study was conducted with the formulation 3A (Surelease/Opadry). The in-use study consisted of sprinkling the contents of two capsules of the test product (Quinine Sulfate Capsules, 324 mg (Surelease/Opadry)) on 15 mL of applesauce. After 1 hour the mixture was assayed for quinine using a validated method. The results indicated that the assay of the product when sprinkled on applesauce for 1 hour (99.9%) was similar to the assay of the contents of two capsules without applesauce (101.1%). Thus, the sprinkled applesauce may be prepared and stored up to 1 hour before ingestion since the formulation of quinine is chemically stable for up to 1 hour on applesauce.

### Example 5b

### Palatability Study for a 7-day, 3 Times a Day (TID) Dosing Regimen

A Five-Way Cross-Over Study was used to evaluate the palatability of a single 648-mg dose (2×324 mg capsules) of the quinine sulfate capsule formulation of Example 3A (Surelease/Opadry) compared to a single 648-mg dose (2×324 mg capsules) of Quinine Sulfate Capsules USP (Qualaquin®) when sprinkled on sweetened applesauce. The study is also used to evaluate likelihood of completing a 7-day three times a day (TID) regimen of formulation 3A (Surelease/Opadry) compared to quinine sulfate capsules USP (Qualaquin®) when sprinkled on sweetened applesauce.

The study consisted of 5 single-day study periods. Within five minutes of completing a standard breakfast, each subject receives test (Treatments A-D, formulation 3A) or reference (Treatment E, Qualaquin®) products, followed with 240 mL of water, in a randomly-assigned sequence, with each treatment separated by at least a 3-day washout period:

Treatment A: Quinine-sulfate capsule formulation 3A (Surelease/Opadry), single 648 mg dose (2×324 mg) capsules opened and sprinkled on one tablespoon (15 mL) sweetened applesauce (Musselman®) ingested immediately;

Treatment B: Quinine-sulfate capsule formulation 3A (Surelease/Opadry), single 648 mg dose (2×324 mg) capsules opened and sprinkled on one tablespoon (15 mL) sweetened applesauce (Musselman®) for 15 minutes prior to ingestion;

Treatment C: Quinine-sulfate capsule formulation 3A (Surelease/Opadry), single 648 mg dose (2×324 mg) capsules opened and sprinkled on one tablespoon (15 mL) sweetened applesauce (Musselman®) for 30 minutes prior to ingestion;

Treatment D: Quinine-sulfate capsule formulation 3A (Surelease/Opadry), single 648 mg dose (2×324 mg) capsules opened and sprinkled on one tablespoon (15 mL) sweetened applesauce (Musselman®) for 60 minutes prior to ingestion;

Treatment E: Quinine-sulfate capsule USP (Qualaquin®), single 648 mg dose (2×324 mg) capsules opened and sprinkled on one tablespoon (15 mL) sweetened applesauce (Musselman®) ingested immediately.

Subjects receive a single oral dose of the study medication at each treatment period visit. Within 5 minutes after dosing the subject's verbal response to a palatability question is elicited. The subject is shown a horizontally oriented sequence of integers from “0” to “10” using a 10 cm visual analog scale (VAS). The numbers are spaced at uniform intervals of 10 mm. The scale is anchored by “Not at all difficult” (0) and “Extremely difficult” (10). The palatability question consists of “Users of this product will be required to take it once every 8 hours (3 times per day) for 7 days. Now, please think about the (taste—immediately after dosing) (aftertaste—15 minutes or 1 hour after dosing) of the product you have just taken and use this 0 to 10 scale to tell me how difficult it would be for you to complete this 7-day treatment. Zero would mean that you would have no difficulty at all. Ten would mean that you would find it extremely difficult. The larger the number you select the more difficult you think it will be.” The result of the palatability study is provided in the table below.

Mean Palatability VAS Scores for Treatments A to E

| | | | Palatability Score | | |
|---|---|---|---|---|---|
| | | # of | Mean | | |
| | Time | subjects, N | (SD) | Median | Range |
| Treatment A | immediately | 29 | 0.6 (1.6) | 0.0 | 0-8 |
| Formulation | | | | | |
| 3A | | | | | |
| Treatment B | 15 minutes | 29 | 0.4 (1.2) | 0.0 | 0-6 |
| Formulation | | | | | |
| 3A | | | | | |
| Treatment C | 30 minutes | 29 | 1.2 (2.2) | 0.0 | 0-7 |
| Formulation | | | | | |
| 3A | | | | | |
| Treatment D | 60 minutes | 28 | 2.4 (2.9) | 1.0 | 0-10 |
| Formulation | | | | | |
| 3A | | | | | |
| Treatment E | immediately | 29 | 8.6 (2.1) | 10.0 | 3-10 |
| Qualaquin ® | | | | | |

As indicated by the study results, subjects indicated a significantly greater likelihood to complete the 7-day TID regimen of formulation 3A when sprinkled on sweetened applesauce for up to 60 minutes prior to consumption compared to Qualaquin® when sprinkled on sweetened applesauce and consumed immediately (all adjusted P-values were <0.0001). For formulation 3A, subjects indicated no statistically significant differences in the likelihood of completing the 7-day TID regimen if the contents were sprinkled and left on the sweetened applesauce for 0 minutes (immediately), 15 minutes, or 30 minutes prior to consumption. Although statistically significant differences were noted for 0, 15, and 30 minutes compared to 60 minutes, the median of intra-subject differences in the VAS scores were small (<1.0). This indicates an acceptable palatability for the contents of the formulation 3A to be sprinkled on applesauce for up to 60 minutes.

### Examples 6-12

### Bioavailability and Food-Effect Studies

### Example 6

### Relative Bioavailability Under Fasting Conditions of a Formulation of Example 3 in Comparison to Qualaquin® and Food-Effect Evaluation

A three-way crossover study is used to evaluate the pharmacokinetic parameter values of a single 648-mg dose (2×324 mg capsules) of the quinine sulfate capsule formulations of Example 3C (CAP/HPMC—10% weight gain) as compared to the pharmacokinetics of a single 648 mg dose (2×324 mg capsules) of Qualaquin® under fasted conditions. The study is also used to evaluate the effect of food (a high-fat breakfast) on the pharmacokinetics of a single 648-mg dose (2×324 mg capsules) of the quinine sulfate capsule formulations of Example 3C (CAP/HPMC—10% weight gain) when administered with a high-fat breakfast as compared to a single 648 mg dose (2×324 mg capsules) of the same formulation administered under fasted conditions in healthy adult volunteers.

The quinine concentration-time data are used to calculate the following pharmacokinetic parameters: AUC₀₋ₜ, AUC_{0-∞}, Cₘₐₓ, and Tₘₐₓ. The pharmacokinetic parameters are evaluated statistically by an analysis of variance (ANOVA) appropriate for the experimental design of the study. Analyses for AUC₀₋ₜ, AUC_{0-∞}, and Cₘₐₓ are performed on ln-transformed data. For ln-transformed AUC₀₋ₜ, AUC_{0-∞}, and Cₘₐₓ, estimates of the adjusted differences between treatment means and the standard error associated with these differences are used to construct a 90% confidence interval for the ratio of the test to reference population means. The results are provided in Tables 19-20.

### Examples 7-8

Similar three-way studies as Example 6 are performed with the formulation of Example 3A compared to Qualaquin® (Example 7) and the formulation of Example 3E compared to Qualaquin® (Example 8). The results are provided in Tables 19-20.

### Example 9

In another study, a single-dose, open-label, randomized, three-period, three-treatment crossover, is used to evaluate the pharmacokinetic parameter values of a single 648-mg dose (2×324 mg capsules) of the quinine sulfate capsule formulation of Example 3F (EPO—6% weight gain) and Example 3G (EPO—20% weight gain) as compared to the pharmacokinetics of a single 648 mg dose (2×324 mg capsules) of Qualaquin® under fasted conditions in healthy adult volunteers. Blood samples are drawn up to 24 hours post dose and evaluated as in Example 6. The results are provided in Table 19.

### Example 10

A similar three-way study as in Example 9 is performed with the quinine sulfate capsule formulations of Examples 3H and 3I compared to Qualaquin®. The results are provided in Table 19.

### Example 11

In yet another study, a randomized, single dose, two-arm, two-way crossover, single dose food-effect study is performed with the quinine sulfate capsule formulation of Example 3F. During each study period, all subjects are randomized to receive a single 648-mg dose (2×324-mg capsules) of the Example 3F formulation (6% EPO) following a minimum overnight fast of 10 hours, or a single 648 mg dose (2×324-mg capsules) of the Example 3F formulation (6% EPO) within 5 minutes of completing a standard, high-fat breakfast; subjects will have 30 minutes to complete the entire breakfast. There will be a 7-day washout period between treatments. Blood samples are drawn up to 48 hours post dose and evaluated as in Example 6. The results are provided in Table 20.

### Example 12

A similar two-way study as in Example 11 is performed with the quinine sulfate capsule formulation of Example 3H with an additional blood sample drawn at 72 hours post dose. The results are provided in Table 20.

TABLE 19
Test formulations v. Qualaquin ®; Fasted

| | | | | 90% Confidence Interval | | |
|---|---|---|---|---|---|---|
| | % Ratio | | | (Lower Limit, Upper Limit) | | |
| Formulation | Cₘₐₓ | AUC₀₋ₜ | AUC_{0-∞} | Cₘₐₓ | AUC₀₋ₜ | AUC_{0-∞} |
| 3F; 6% EPO | 93 | 98 | 101 | (86, 101) | (92, 104) | (94, 109) |
| coating | | | | | | |
| N = 23 | | | | | | |
| 3G; 20% EPO | 91 | 94 | 101 | (84, 99) | (89, 99) | (94, 108) |
| coating | | | | | | |
| N = 23 | | | | | | |
| 3H; 6% | 34 | 64 | 67 | (30, 39) | (56, 73) | (58, 76) |
| L30D55 | | | | | | |
| N = 23 | | | | | | |
| 3I; 12% | 35 | 65 | 67 | (30, 40) | (57, 74) | (58, 76) |
| L30D55 | | | | | | |
| N = 23 | | | | | | |
| 3A; | 102 | 99 | 98 | (96, 107) | (94, 105) | (92, 105) |
| Surelease | | | | | | |
| Opadry | | | | | | |
| N = 29 | | | | | | |
| 3C; 10% CAP/ | 98 | 103 | 103 | (93, 104) | (97.5, 109) | (97, 110) |
| HPMC | | | | | | |
| N = 28 | | | | | | |
| 3D; 20% CAP/ | 89 | 95 | 95 | (85, 94) | (89, 101) | (89, 102) |
| HPMC | | | | | | |
| N = 26 | | | | | | |
| 3E; 6% EPO | 96 | 96 | 96 | (91, 102) | (91, 101) | (91, 101) |
| micronized | | | | | | |
| N = 28 | | | | | | |

As shown by the bioavailability study results in Table 19, the quinine sulfate capsule formulations of Examples 3A, 3C, 3D, 3E, 3F and 3G are bioequivalent to Qualaquin® under fasting conditions.

TABLE 20
Fed v. Fasted

| | | | | 90% Confidence Interval | | |
|---|---|---|---|---|---|---|
| | % Ratio | | | (Lower Limit, Upper Limit) | | |
| Formulation | Cₘₐₓ | AUC₀₋ₜ | AUC_{0-∞} | Cₘₐₓ | AUC₀₋ₜ | AUC_{0-∞} |
| Qualaquin ® | 104 | 104 | 104 | (99, 110) | (101, 106) | (100, 107) |
| 3F; 6% EPO | 109 | 102 | 102 | (103, 115) | (98, 107) | (97, 107) |
| coating | | | | | | |
| N = 30 | | | | | | |
| 3H; 6% | 168 | 150 | 149 | (151, 188) | (132, 171) | (129, 172) |
| L30D55 | | | | | | |
| N = 28 | | | | | | |
| 3A; | 95 | 107 | 109 | (90, 100) | (101, 114) | (102, 116) |
| Surelease/ | | | | | | |
| Opadry | | | | | | |
| N = 29 | | | | | | |
| 3C; 10% CAP/ | 106 | 98 | 98 | (100, 112) | (93, 104) | (92, 105) |
| HPMC | | | | | | |
| N = 28 | | | | | | |
| 3D; 20% CAP/ | 121 | 108 | 106 | (115, 127) | (102, 115) | (99, 114) |
| HPMC | | | | | | |
| N = 26 | | | | | | |
| 3E; 6% EPO | 109 | 107 | 106 | (103, 116) | (102, 112) | (101, 112) |
| micronized | | | | | | |
| N = 28 | | | | | | |

As shown by the food-effect study results in Table 20, the quinine sulfate capsule formulations of Examples 3A, 3C, 3D, 3E, and 3F are bioequivalent to Qualaquin® under non-fasting conditions.

### Example 13

### Relative Bioavailability Under Fasting Conditions of a Formulation of Example 3a in Comparison to Qualaquin® Capsules and Food-Effect Evaluation (High Fat Breakfast) and Sprinkled in Sweetened Applesauce in Healthy Adult Volunteers

A four-way crossover study is used to evaluate the pharmacokinetic parameter values of a single 648-mg dose (2×324 mg capsules) of the quinine sulfate capsule formulation of Example 3A (Surelease/Opadry) (N=46) under fasted conditions as compared to the pharmacokinetics of a single 648 mg dose (2×324 mg capsules) of Qualaquin® under fasted conditions (N=42). The study is also used to evaluate the effect of food (a high-fat breakfast) on the pharmacokinetics of a single 648-mg dose (2×324 mg capsules) of the quinine sulfate capsule formulation of Example 3A (Surelease/Opadry) when administered with a high-fat breakfast (N=43) as compared to a single 648 mg dose (2×324 mg capsules) of the same formulation administered under fasted conditions (N=46). The study is also used to evaluate the effect on the pharmacokinetics of a single 648-mg dose (2×324 mg capsules' contents) of the quinine sulfate capsule formulation of Example 3A (Surelease/Opadry) when administered as a sprinkle in 15 ml of sweetened applesauce (N=44) as compared to a single 648-mg dose (2×324 mg capsules) of the quinine sulfate capsule formulation of Example 3A (Surelease/Opadry) (N=46) under fasted conditions. The pharmacokinetic parameters are evaluated statistically as previously described. The results are provided in Table 21.

TABLE 21

| | | | | 90% Confidence Interval | | |
|---|---|---|---|---|---|---|
| | % Ratio | | | (Lower Limit, Upper Limit) | | |
| Formulation | Cₘₐₓ | AUC₀₋ₜ | AUC_{0-∞} | Cₘₐₓ | AUC₀₋ₜ | AUC_{0-∞} |
| 3A; Surelease/ | 96 | 99 | 98 | (90, 102) | (92, 105) | (94, 103) |
| Opadry fasted: | | | | | | |
| Qualaquin ® | | | | | | |
| fasted | | | | | | |
| 3A; Surelease/ | 102 | 105 | 105 | (96, 109) | (98, 112) | (101, 110) |
| Opadry | | | | | | |
| fed: fasted | | | | | | |
| 3A; Surelease/ | 97 | 97 | 102 | (91, 103) | (91, 103) | (97, 106) |
| Opadry | | | | | | |
| Sprinkled: 3A; | | | | | | |
| Surelease/ | | | | | | |
| Opadry fasted | | | | | | |

As the results show in Table 21, the quinine sulfate capsule formulation of Example 3A is bioequivalent to Qualaquin® under fasting conditions. Furthermore, the quinine sulfate capsule formulation of Example 3A under fed conditions is bioequivalent to the same formulation under fasting conditions. Finally, quinine sulfate formulation of Example 3A, when administered as a sprinkle on applesauce, is bioequivalent to the same formulation administered as a capsule under fasting conditions.

### Example 14

### Study to Investigate the Pharmacokinetics and QT Interval Prolongation of Quinine Sulfate in Healthy Young and Elderly Subjects

A three-way crossover study is used to investigate the pharmacokinetics and QT interval prolongation of quinine sulfate in healthy young (≧18 to ≦45 years) and elderly (≧65 to ≦80 years) subjects. Sixteen young and fifteen elderly are used in the study. Objectives of the study included comparison of the pharmacokinetics of quinine after multiple oral doses of formulation 3A capsules in healthy, young and elderly, male and female subjects; to investigate the effect of quinine on ventricular repolarization, as measured by prolongation of the QT interval, in healthy, young and elderly, male and female subjects; to evaluate the correlation between changes in delta QTc and quinine plasma concentrations in healthy, young and elderly, male and female subjects; and to evaluate the safety and tolerability of formulation 3A capsules in healthy, young and elderly, male and female subjects.

Each subject receives each of the following three treatments beginning on Day 1 of the treatment period, in a randomly assigned sequence, with each treatment period separated by a minimum 4-day washout between study periods:
Treatment A, A 648-mg dose of quinine sulfate capsules, 2×324 mg capsules formulation 3A administered orally three times per day (every 8 hours) on Days 1 to 6 with a single dose on the morning of Day 7.
Treatment B Placebo capsule (2) administered orally three times per day (every 8 hours) on Days 1 to 6 with a single dose on the morning of Day 7.
Treatment C Placebo capsule (2) administered orally three times per day (every 8 hours) on Days 1 to 6 then a single dose of moxifloxacin (400 mg) on the morning of Day 7.

Subjects were to receive the morning dose of assigned treatment 30 minutes prior to the morning meal.

Supine blood pressure (BP) and heart rate (HR) are measured prior to the first dose of study medication every day in each study period. On the last dosing day of each period (Day 7), vital signs are repeated at 2, 6, and 12 hours post dosing and again at 24-hours (post-Day 7 dose [Day 8]) and 48 hours (post-Day 7 dose [Day 9]). Triplicate baseline electrocardiograms (ECGs) are collected by Holter monitor at 1.0, 0.5, and 0.25 hours prior to the first dose of each study period. Continuous Holter monitoring is performed prior to the last dose on Day 7 of each study period and continue for 48 hours post-dose (ending the morning of Day 9 of each study period). Subjects rest for at least 10 minutes prior to the designated time points for ECGs. Over the 24-hour period on Days 7 and 8 of each study period, triplicate ECGs are collected from the Holter monitor data over a 5-minute period within approximately 10 minutes of each specified time point at pre-dose and 0.5, 1, 2, 2.5, 3, 4, 6, 8, 12, 14, 24, and 48 hours post-dose.

The results of the study showed that quinine increased the Rate-corrected QT interval using individual exponent formula (“QtcI”) above 20 msec over placebo for the first 6 hours and above 10 msecs over placebo up to 14 hours after dosing. The maximal increase over placebo was 28.3 msec and the upper bound of the one sided confidence interval was 31.83 msec one hour post dose. QTcI differences between the elderly and young between quinine and placebo (baseline adjusted) ranged from 10.06 msec at 30 minutes post dose to −1.92 msec 48 hours post dose, with a mean increase of 3.92 msec over 48 hours. These differences were well within the anticipated increase of Rate-corrected QT interval (“QTc”) interval with advancing age. No subject had a QTcI greater than 500 msec or had a maximum QTcI change from baseline of greater than 60 msec. QT interval on the ECG (“QT”) results are provided in Table 22 below by age.

TABLE 22
Mean Time-Matched Differences from Placebo in Changes from Predose
Baseline in QTcI for Quinine and Placebo by Age Group (msec)

| Hour (Post-dose) | Young (N = 16) | Elderly (N = 15) | P-value* |
|---|---|---|---|
| 0.5 | 19.10 | 29.16 | 0.064 |
| 1.0 | 25.79 | 30.20 | 0.484 |
| 2.0 | 24.98 | 28.92 | 0.586 |
| 2.5 | 26.35 | 38.41 | 0.745 |
| 3.0 | 24.55 | 29.75 | 0.391 |
| 4.0 | 23.40 | 28.27 | 0.380 |
| 6.0 | 18.19 | 24.77 | 0.230 |
| 8.0 | 17.67 | 19.87 | 0.633 |
| 12.0 | 12.86 | 14.45 | 0.711 |
| 14.0 | 11.16 | 11.42 | 0.952 |
| 24.0 | 3.52 | 11.28 | 0.074 |
| 48.0 | −0.11 | −2.03 | 0.708 |

*Age Group-by-treatment group interaction

The study investigating the effect of age on QTc interval prolongation following quinine administration did not indicate that patients ≧65 years of age have significantly greater risk of QTc prolongation than younger patients.

The study also revealed following multiple 648 mg twice-daily oral doses of formulation 3A capsules for 7 days, the mean AUCss was 5% higher in 15 elderly subjects (65 to 78 years old) than in 16 young subjects (20 to 39 years old). The mean steady-state Cmax in elderly subjects (6.75 ug/mL) was slightly higher than in younger subjects (6.42 ug/mL). The mean oral clearance was similar in elderly and young subjects (0.195 vs 0.192 L/h/kg, respectively). The mean elimination half-life in the elderly subjects (25.2 hours) was longer than in younger subjects (20.1 hours). Statistically significant differences in the rate and extent of absorption (approximately 25% increase) and elimination half-life (15% increase) of the major metabolite, 3-hydroxyquinine, were seen in the elderly subjects as compared to the younger subjects, Table 23.

TABLE 23
Summary of Statistical Analysis for Age Differences at Steady State
(Days 7-9)

| | Least Squares Mean | | | | |
|---|---|---|---|---|---|
| PK | ≧65 to | ≧18 to | | 90% | |
| Parameter | ≦80 yrs | ≦45 yrs | % | Confidence | p- |
| (unit) | (N = 15) | (N = 16) | Ratio | Interval | value |

Quinine

| | | | | | |
|---|---|---|---|---|---|
| Cₘₐₓ₍ₛₛ₎ | 6.75 | 6.4 | 105.17 | 90.75, 119.59 | 0.5470 |
| (μg/mL) | | | | | |
| AUC₍ₛₛ₎ | 48.2 | 46.1 | 104.56 | 90.01, 119.11 | 0.5983 |
| (μg · hr/mL) | | | | | |
| Tₘₐₓ₍ₛₛ₎ (hr) | 2.00 | 2.16 | 92.67 | 67.28, 118.06 | 0.6276 |
| T_{1/2} (hr) | 25.2 | 20.1 | 125.29 | 105.57, 145 | 0.0376 |

3-OH Quinine

| | | | | | |
|---|---|---|---|---|---|
| Cₘₐₓ₍ₛₛ₎ | 1.95 | 1.56 | 125.30 | 105.01, | 0.0428 |
| (μg/mL) | | | | 145.59 | |
| AUC₍ₛₛ₎ | 14.1 | 11.1 | 126.88 | 106.3, 147.45 | 0.0344 |
| (μg · hr/mL) | | | | | |
| Tₘₐₓ₍ₛₛ₎ (hr) | 1.60 | 1.53 | 104.58 | 66.03, 142.7 | 0.8480 |
| T_{1/2} (hr) | 18.7 | 16.2 | 114.96 | 102.79, | 0.0456 |
| | | | | 127.12 | |

### Example 15

### Micronizing Quinine Sulfate

Quinine sulfate is micronized using a Fluid Energy Jet Mill subclass Tangential Jet, mill size 8-inch Fluid Energy Jet Mill constructed of 316 L stainless steel. Particle size reduction is achieved through impact and attrition due to high velocity collisions between particles suspended within the air stream, causing them to break down into smaller particles. Particle size distribution is measured on a Malvern Mastersizer 2000 particle size analyzer using hexanes as the dispersant. Particle size specification is D(v,0.9) of less than 10 micrometers.

The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”). The endpoints of all ranges directed to the same component or property are inclusive and independently combinable.

A “patient” means a human or non-human animal in need of medical treatment. Medical treatment can include treatment of an existing condition, such as a disease or disorder, prophylactic or preventative treatment, or diagnostic treatment. In some embodiments the patient is a human patient. The terms “treating” and “treatment” mean implementation of therapy with the intention of reducing in severity or frequency symptoms, elimination of symptoms or underlying cause, prevention of the occurrence of symptoms or their underlying cause, and improvement or remediation of damage.

By an “effective” amount or a “therapeutically effective amount” of an active agent is meant a sufficient amount of the active agent to produce a therapeutic effect in the patient. The amount that is “effective” will vary from subject to subject, depending on the age and general condition of the individual, the particular active agent, and the like. Thus, it is not always possible to specify an exact “effective amount.” However, an appropriate “effective” amount in any individual case may be determined by one of ordinary skill in the art using routine experimentation.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or

[symbol]

1. A quinine sprinkle formulation, comprising:
a plurality of coated subunits, wherein each coated subunit comprises
a core subunit comprising quinine or a pharmaceutically acceptable salt thereof, and a pharmaceutically acceptable excipient, and
a coating on the outside of the core subunit, wherein the coating comprises a polymeric coating material, wherein the polymeric coating material is chitosan; ethylcellulose; hydroxypropyl methylcellulose acetate succinate; cellulose acetate phthalate; a (meth)acrylic acid copolymer; hydroxypropyl methylcellulose succinate; cellulose acetate succinate; cellulose acetate hexahydrophthalate; hydroxypropyl methylcellulose hexahydrophthalate; hydroxypropyl methylcellulose phthalate; cellulose propionate phthalate; cellulose acetate maleate; cellulose acetate trimellitate; cellulose acetate butyrate; cellulose acetate propionate; a polyvinylacetate phthalate; zein; or a combination thereof; optionally in combination with a plasticizer, a stabilizer, a water-soluble component, an anti-tacking agent, a surfactant, or a combination thereof;
wherein the quinine formulation exhibits immediate-release profile; and
wherein the quinine sprinkle formulation forms a palatable mixture with applesauce for up to one hour after adding the plurality of coated subunits in applesauce.

2. The quinine sprinkle formulation of claim 1, wherein the polymeric coating material is a combination of ethylcellulose and hydroxypropyl methylcellulose, a combination of cellulose acetate phthalate and hydroxypropyl methylcellulose; or a poly(methacrylic acid, ethyl acrylate) 1:1.

3. The quinine sprinkle formulation of claim 1, wherein the coated subunit comprises about 1 to about 30% weight gain polymeric coating material and optional water-soluble component based on the total weight of the core subunit, polymeric coating material, and water-soluble component.

4. The quinine sprinkle formulation of claim 1, wherein the coated subunit comprises about 1 to about 7% weight gain polymeric coating material and water-soluble component based on the total weight of the core subunit, polymeric coating material, and water-soluble component; and
wherein the polymeric coating material comprises ethylcellulose and the water-soluble component comprises hydroxypropyl methylcellulose,
wherein the ethylcellulose and hydroxypropyl methylcellulose are in a weight ratio of about 2:1 to about 1:2.

5. The quinine sprinkle formulation of claim 1, wherein the coated subunit comprises about 2 to about 6% weight gain polymeric coating material and water-soluble component based on the total weight of the core subunit, polymeric coating material, and water-soluble component; and
wherein the polymeric coating material comprises ethylcellulose and the water-soluble component comprises hydroxypropyl methylcellulose,
wherein the ethylcellulose and hydroxypropyl methylcellulose are in a weight ratio of about 1.5:1 to about 1:1.5.

6. The quinine sprinkle formulation of claim 1, wherein the coated subunit comprises about 6 to about 14% weight gain polymeric coating material and water-soluble component based on the total weight of the core subunit, polymeric coating material, and water-soluble component; and
wherein the polymeric coating material comprises cellulose acetate phthalate and the water-soluble component comprises hydroxypropyl methylcellulose,
wherein the cellulose acetate phthalate and hydroxypropyl methylcellulose are in a weight ratio of about 3:1 to about 1:1.

7. The quinine sprinkle formulation of claim 1, wherein the coated subunit comprises about 8 to about 12% weight gain polymeric coating material and water-soluble component based on the total weight of the core subunit, polymeric coating material, and water-soluble component; and
wherein the polymeric coating material comprises cellulose acetate phthalate and the water-soluble component comprises hydroxypropyl methylcellulose,
wherein the cellulose acetate phthalate and hydroxypropyl methylcellulose are in a weight ratio of about 2.6:1 to about 2:1.

8. The quinine sprinkle formulation of claim 1, wherein the coated subunit comprises about 4 to about 20% weight gain polymeric coating material based on the total weight of the core subunit and polymeric coating material, wherein the polymeric coating material comprises a poly(methacrylic acid, ethyl acrylate) 1:1.

9. The quinine sprinkle formulation of claim 1, wherein the sprinkle formulation is a capsule comprising a plurality of coated subunits totaling about 324 mg quinine sulfate per capsule.

10. The quinine sprinkle formulation of claim 1, wherein the quinine sprinkle formulation exhibits a dissolution profile such that after combining the formulation with 900 ml of 0.1N HCl, optionally containing pepsin (activity of pepsin between 607,500 to 750,000 Units per liter of dissolution medium), at 37° C.±0.5° C. using a tablet dissolution apparatus equipped with a basket stirring element, 100 rpm shaft speed, greater than or equal to 80% of the active agent is released within 60 minutes.

11. The quinine sprinkle formulation of claim 1, wherein
wherein the quinine sprinkle formulation leaches less than 0.6% quinine as determined by reverse-phase High Performance Liquid Chromatography (HPLC) analysis on a sample taken at 10 minutes from the time the formulation is mixed with four ounces of unsweetened applesauce as a sprinkle;
wherein the HPLC analysis is performed using a reverse-phase column at a column temperature of about 30° C.; a flow rate of 0.5 mL/minute; injection volume of 10 μA; detection at 249 nm; and mobile phase of 10 mM Ammonium Bicarbonate Buffer pH 9.5:Acetonitrile:Methanol (650:300:50); and
wherein the sample for the HPLC analysis comprises weighing a five gram aliquot of the applesauce ensuring no subunit is included in the aliquot; adding about 30 ml diluent (10 mM Ammonium Bicarbonate Buffer pH 9.5:Acetonitrile:Methanol (650:300:50)); shaking the flask for 15 minutes using a wrist action shaker; adding diluent to result in 50 ml volume; mixing; centrifuging a portion of the prepared sample at 3000 rpm for 15 minutes; and testing the supernatant by reverse-phase HPLC analysis.

12. A method of administering quinine, comprising:
administering a quinine sprinkle formulation to a patient for treatment of uncomplicated Plasmodium falciparum malaria, treatment of severe or complicated Plasmodium falciparum malaria, treatment of Plasmodium vivax infection, treatment of babesiosis caused by Babesia microti, or prevention of malaria;
wherein the quinine sprinkle formulation comprises a plurality of coated subunits, wherein each coated subunit comprises
a core subunit comprising quinine or a pharmaceutically acceptable salt thereof, and a pharmaceutically acceptable excipient, and
a coating on the outside of the core subunit, wherein the coating comprises a polymeric coating material, wherein the polymeric coating material is chitosan; ethylcellulose; hydroxypropyl methylcellulose acetate succinate; cellulose acetate phthalate; a (meth)acrylic acid copolymer; hydroxypropyl methylcellulose succinate; cellulose acetate succinate; cellulose acetate hexahydrophthalate; hydroxypropyl methylcellulose hexahydrophthalate; hydroxypropyl methylcellulose phthalate; cellulose propionate phthalate; cellulose acetate maleate; cellulose acetate trimellitate; cellulose acetate butyrate; cellulose acetate propionate; a polyvinylacetate phthalate; zein; or a combination thereof; optionally in combination with a plasticizer, a stabilizer, a water-soluble component, an anti-tacking agent, a surfactant, or a combination thereof;
wherein the quinine formulation exhibits immediate-release profile; and
wherein the quinine sprinkle formulation is a palatable mixture for up to one hour after preparation of the mixture, the palatable mixture comprising the plurality of coated subunits sprinkled in applesauce.

13. A method of administering quinine for treatment of uncomplicated Plasmodium falciparum malaria, treatment of severe or complicated Plasmodium falciparum malaria, treatment of Plasmodium vivax infection, treatment of babesiosis caused by Babesia microti, or prevention of malaria, comprising:
administering to a patient in need of quinine therapy an immediate release quinine sprinkle dosage form of claim 1 as a palatable mixture for up to one hour after preparation of the mixture, the palatable mixture comprising the plurality of coated subunits sprinkled in applesauce.

14. The method of claim 13, where the patient finds the mixture palatable, wherein palatability is measured using a 10 cm visual analog scale and the mean palatability score is below 3.

15. The method of claim 13, wherein 648 mg quinine sulfate is administered for 7 days TID.
